(19) United States
(12) Patent Application Publication
(10) Pub. No.: US 2019/0255299 A1
(43) Pub. Date: Aug. 22, 2019

(54) INTRAVASCULAR DELIVERY SYSTEM AND METHOD FOR PERCUTANEOUS CORONARY INTERVENTION

(71) Applicant: CROSSLINER, LLC, Kalamazoo, MI (US)

(72) Inventors: TIM A. FISCHELL, KALAMAZOO, MI (US); FRANK S. SALTIEL, WILLOWBROOK, IL (US)

(21) Appl. No.: 16/132,878

(22) Filed: Sep. 17, 2018

Related U.S. Application Data
(63) Continuation-in-part of application No. 15/899,603, filed on Feb. 20, 2018.

(51) Int. Cl. A61M 25/10; A61F 2/958; A61F 2/966; A61M 25/00

(52) U.S. Cl. CPC A61M 25/104; A61F 2/958; A61F 2/966; A61M 2025/1081; A61M 2205/0266; A61M 2025/1079; A61F 2240/001; A61M 25/0012

(57) ABSTRACT

The subject guide catheter extension/pre-dilatation system includes an outer delivery sheath, an inner member extending within the sheath, and a mechanism for engagement/disengagement of the inner member to/from the sheath. The inner member is configured with a tapered distal tip having a delivery micro-catheter and a pre-dilatation balloon member attached to the tapered distal tip. The guidewire and a guide catheter are advanced to the vicinity of the treatment site within a blood vessel. Subsequently, the inner member and outer delivery sheath, in their engaged configuration, are advanced along the guidewire inside the guide catheter towards the site of treatment. At the treatment site, the balloon member is inflated for pre-dilatation treatment. The inner member is disengaged and retracted from the outer delivery sheath, and a stent is delivered to the treatment site.

### REFERENCE TO RELATED APPLICATIONS

The present Utility patent application is a Continuation-in-Part (CIP) of the Utility patent application Ser. No. 15/899,603, filed on 20 Feb. 2018, currently pending.

### INCORPORATION BY REFERENCE

U.S. patent application Ser. No. 15/899,603, currently pending, is hereby incorporated by reference.

### FIELD OF THE INVENTION

The present invention is directed to medical devices, and, in particular, to minimally invasive devices used for treatment within the human vasculature, such as, for example, coronary arteries.

More in particular, the present invention addresses a delivery system for percutaneous coronary intervention adapted specifically for intravascular balloon angioplasty, and enhanced by pre-dilatation guide catheter extension capabilities.

The present invention is also directed to medical devices designed for atraumatic, convenient and fast delivery of various interventional devices, such as, for example, a pre-dilatation balloon, or stents, and replacement of catheters in coronary arteries (or other blood vessels) in a patient body to facilitate percutaneous revascularization.

Furthermore, the present invention is directed to a pre-dilatation balloon delivery arrangement releasably integrated with an outer delivery sheath, and equipped with a distal tapered micro-catheter sliding on a guide wire that facilitates practically atraumatic crossability of a pre-dilatation balloon and the outer delivery sheath to a site of a lesion for treatment.

In overall novel concept, the present invention is directed to an intravascular delivery system configured with an outer delivery sheath sub-system and an interventional device delivery sub-system cooperating with the outer delivery sheath sub-system, where the interventional device delivery sub-system is equipped with a highly flexible tapered elongated delivery micro-catheter positioned at the distal end of the interventional device delivery sub-system and fitted within the outer delivery sheath sub-system with the distal end of the interventional device delivery sub-system fixed at a predetermined position beyond the distal end of the outer delivery sheath and prevented from forward displacement relative thereto. The subject system is specifically designed to track over a guide wire to deliver the interventional device (such as a dilatation balloon, or a stent, etc.) attached in proximity to the micro-catheter to a site of interest in a diseased blood vessel.

The subject invention further addresses an intravascular delivery system which has a miniature profile with a diameter not exceeding 1 mm at its distal end, and capable of an interventional device deliverability that would be superior to that of the conventional balloon angioplasty catheters.

The present invention is also directed to an intravascular guide catheter extension/pre-dilatation system using an inner member (interventional device delivery sub-system) positioned at a predetermined location internally of an outer member (the outer delivery sheath sub-system), where the inner member is formed with a tapered portion interfacing with a slightly tapered distal end of the outer member, such that there is a virtually “seamless” transition on the interface between the inner and outer members which is highly beneficial for an atraumatic and smooth passage of the inner and outer member as a single unit along a diseased blood vessel.

Furthermore, the present invention is directed to an intravascular guide catheter extension/pre-dilatation system designed with an interconnection (locking) mechanism which is actuated/de-actuated by a physician to either controllably engage the inner and outer members for the integral motion within a guide catheter along a guide wire, or disengage the inner and outer members for retraction of the inner member from the outer member, as required by the intravascular procedure, where the inner member carries an interventional device (such as a pre-dilatation balloon member, or a stent) attached at its tapered distal end in proximity to a tapered micro-catheter formed thereat.

Additionally, the present invention is directed to an intravascular guide catheter extension/pre-dilatation system which is configured with a tapered shaft at its distal end for carrying the balloon member thereon and which provides a “seamless” entry and smooth deliverability of the balloon member integral with the outer delivery sheath to the treatment site.

### BACKGROUND OF THE INVENTION

Coronary artery obstruction disease, or a disease in the peripheral vasculature, is often treated by the balloon angioplasty and/or stent placement. The advancement of the revascularization devices, such as balloons or stent delivery systems, within the blood vessels to a treatment site can be challenging in case of tortuosity and/or calcification of the vessels.

A coronary stent is a tube-shaped device placed in the coronary arteries that supply blood to the heart, to keep the arteries open in the treatment of coronary heart disease. It is used in a procedure called Percutaneous Coronary Intervention (PCI). Stents reduce chest pain and have been shown to improve survivability in the event of an acute myocardial infarction.

Treating a blocked coronary artery with a stent follows the same steps as other angioplasty procedures with important differences. The compressed stent mounted on a balloon significantly reduces the flexibility of the balloon and compromises its smooth advancement through the coronary artery. This can make the stent difficult or impossible to reach a treatment site and risks dislodgement of the un-deployed stent off of its delivery balloon.

Intravascular imaging may be used to assess the lesion's thickness and hardness (calcification) which will affect the deliverability of the stent. A cardiologist uses this information to decide whether to treat the lesion with a stent and if so, what kind and size. Stents, both bare metal and drug-eluting, are most often sold as a unit, with the stent in its collapsed form attached to the outside of a balloon catheter.

Physicians may perform “direct stenting”, where the stent is threaded through the vessel to the lesion and expanded. However, it is common to pre-dilate the blockage before delivering the stent in order to facilitate the stent delivery in more challenging lesions.

Pre-dilatation is accomplished by threading the lesion with an ordinary balloon catheter and expanding it to increase the lesion's diameter. A balloon catheter is a type of “soft” catheter with an inflatable balloon at its tip which is used during a catheterization procedure to enlarge a narrow opening or passage within the body. Subsequent to pre-dilatation, the pre-dilatation balloon is removed, and a stent catheter is threaded through the vessel to the lesion and is expanded, and left as a permanent implant to “scaffold” open the vessel at the lesion site.

Referring to FIGS. 1A, 1B, and 1C, during the stenting procedure, the closed stent 10 is positioned over a balloon 12 which, in its turn, is secured to a distal end of a catheter 14. The catheter 14 is advanced inside the blood vessel 16 to the location of a lesion 18 by sliding over the guidewire 20. As shown in FIG. 1B, when in place, the balloon 12 is inflated and expands the stent 10 to open the blood passage at the place of the lesion 18. As shown in FIG. 1C, the expanded stent compresses the plaque at the lesion site 18 and widens the blood vessel (for example, the artery) so that the blood flow is increased. The balloon 12 along with the catheter 14, and the guidewire 20, are subsequently removed from the blood vessel, while the expanded stent is left at the treatment site, as shown in FIG. 1C.

Balloon catheters used in angioplasty have either over-the-wire (OTW) or rapid exchange (RX) design. Shown in FIGS. 2A and 2B, the balloon catheter slides to the place over the guidewire 20 which can be charged into the balloon catheter through the hub 22 (in the over-the-wire modification shown in FIG. 2A) or through the RX port 24 (for the rapid exchange modification of the balloon catheter, as shown in FIG. 2B). In the over-the-wire balloon catheter, a concentric lumen 26 for passing the guidewire 20 extends within the catheter 14 from the hub 22 to the balloon 27, while in the rapid exchange (RX) balloon catheter, the concentric lumen 28 for the guidewire passage extends from the RX port 24 inside the catheter 14 to the balloon 27 to permit the passage of the guidewire 20.

Revascularization devices usually use guiding (or guide) catheters for delivery of such devices to the site of treatment. The use of guide catheters alone to “back up” the advancement of the revascularization devices to the coronary arteries may be limited and challenging.

In order to facilitate the revascularization devices delivery to the site of interest, guide catheter extension systems have been designed and used during cardiac procedures.

For example, the guide extension system, such as “Guideliner™,” is produced by Teleflex. This guide extension system is described in U.S. Pat. No. 8,292,850, authored by Root, et al. Root, et al. (U.S. Pat. No. 8,292,850) and describes a coaxial guide catheter to be passed through a lumen of a guide catheter, for use with interventional cardiology devices that are insertable into a branch artery that branches off from a main artery.

The Root coaxial guide catheter is extended through the lumen of the guide catheter and beyond its distal end and inserted into the branch artery. Root uses the guide extension supported by a tapered inner catheter. The purpose of the inner catheter is to provide an atraumatic tip to avoid vessel injury, while advancing the guide extension into the proximal portion of a coronary vessel, in order to provide additional “backup” support to deliver the stent or a balloon, especially in a tortuous or calcified artery.

Another guide extension system, such as “Guidezilla™”, has been designed and manufactured by Boston Scientific. This guide extension system is described in U.S. Pat. No. 9,764,118, authored by Anderson, et al. Anderson's guide extension system uses a push member having a proximal portion having a proximal stiffness, a distal portion having a distal stiffness different from the proximal stiffness, and a transition portion disposed and providing a smooth transition between the proximal and distal portions. A distal tubular member is attached to the push member and has an outer diameter larger than the outer diameter of the push member.

U.S. Patent Application Publication #2017/0028178, authored by Ho, describes a guide extension system using a slit catheter which is extendable upon insertion of a balloon or stent delivery system. Ho's guide extension also uses a rigid push rod to assist in delivery of the guide extension to the treatment site.

The systems, “Guideliner™” and “Guidezilla™”, as well as the Ho's system, support the concept of advancing the guide extension system through the guiding catheter, and partially down the coronary artery, in order to achieve additional “back up” support to deliver balloon dilatation catheters and/or stent delivery catheters to the site of intended treatment.

The function of these guide extensions is to permit a closer approach to the lesion to provide additional support in crossing the lesion to be treated with an interventional device. However, despite the additional support, the lesion to be treated can still be difficult or nearly impossible to pass through with a pre-dilatation balloon catheter, or a stent delivery system, due to fibrosis, calcification, and/or angulation at the lesion site.

One of the limitations of the currently used guide extension devices is that they use a relatively blunt and large caliber cylindrical distal end. Relatively high profile distal edges have a limited deliverability of the guide extension in many cases, and permit the advancement only to the proximal or mid portion of the coronary artery to be treated. Very rarely, if ever, can the guide extension be delivered to the actual lesion to be treated with angioplasty or stenting, even after balloon pre-dilatation of the lesion.

U.S. Patent Application Publication #2011/0301502, authored by Gill, describes a catheter with a longitudinal separation, allowing for the positioning device to be smaller in diameter than the stent delivery system. The Gill device, however, does not envision an inner catheter to permit easy and atraumatic crossing of the lesion to be treated. The Gill system acts merely as a covering for the stent delivery system, which can be removed after advancement of the stent delivery system, due to the longitudinal separation.

Although a concept of a tapered piece inside a guide extension catheter is envisioned by Root, the prior art system uses a very short taper, and does not envision the taper as an elongated integrated member of the whole system, nor does it envision that a pre-dilatation balloon can be attached to the tapered delivery micro-catheter to be delivered to the target treatment area. In addition, the prior art fails to envision a substantially “flush” interface between the inner catheter and the outer guide extension inside the vessel, or that the inner and outer catheter members would be reversibly fit or locked together to allow the entire system to be moved easily as one integral device.

Root or other prior art systems do not describe, anticipate or envision a balloon (and/or stent) delivery system, with a very low profile elongated tip which would be beneficial in attaining the coaxial delivery of the guide catheter extension/balloon system to, and beyond, a lesion of interest. Such an embodiment has never been commercialized, and the description of the tapered tip inner device was only meant as a mechanism for the proximal delivery of the blunt tip of the guide catheter extension out of the guiding catheter, but never as a mechanism for delivery of a balloon (and/or stent) to, and beyond, the target treatment area in a blood vessel, nor does it envision that the integral nature, and “flush” interconnection, of the inner and outer members would allow the passage of the outer delivery “sheath” member to cross the lesion of interest.

Thus, a device and method that would permit a delivery of the distal portion of the tubular guide extension system to, or ideally, beyond, the lesion to be treated, would have significant advantages over conventional guide extension devices, such as the “Guideliner™” (Teleflex), or the “Guidezilla™” (Boston Scientific), and others.

Neither of the conventional balloon catheters (over-the-wire or rapid exchange) is integrated with an outer delivery sheath, and neither of them uses a tapered delivery micro-catheter at the distal end of the catheter to which an interventional device (such as the balloon, or stent, etc.) would be secured for atraumatic advancement inside the blood vessel to, and beyond, the lesion site. In addition, neither of the conventional balloon catheters is interconnected with an outer delivery sheath (guide catheter extension sub-system) via an interconnection mechanism actuated to permit integral motion of the conventional balloon catheter and the outer delivery sheath as a single unit, and deactuated to permit retraction of the balloon catheter from the outer delivery sheath, while preventing a forward displacement of the balloon catheter relative the outer delivery sheath.

It would be highly desirable to provide an intravascular delivery system which can deliver an interventional device (for example, a pre-dilatation balloon) along with a guide catheter extension sub-system (such as an outer delivery sheath) to, and beyond, the lesion in a substantially atraumatic and convenient manner.

It would also be highly desirable to facilitate percutaneous revascularization procedures by using a balloon attached to a tapered distal tip of the balloon catheter which would be fitted within the outer delivery sheath serving as a guide catheter extension sub-system, and equipped with a distal elongated tapered micro-catheter at the tapered distal tip to guide an interventional device (the pre-dilatation balloon, and/or stent) to, and past, the lesion to be treated. This would represent substantial improvement upon conventional guide catheter extension and pre-dilatation systems.

### SUMMARY OF THE INVENTION

It is therefore an object of the present invention to provide a medical device for intravascular applications that attains delivery of an interventional device (such as a balloon, or a stent) in an efficient and minimally traumatic fashion, to, and beyond, a coronary artery obstructive lesion by virtue of an integrated distal micro-catheter system.

It is another object of the present invention to provide an intravascular delivery system using a coaxial, highly flexible delivery micro-catheter (which has a diameter at its distal tip not exceeding 1.0 mm), which is specifically configured to track over a 0.009-0.014″ guidewire, and which carries a pre-dilatation balloon attached in close proximity thereto, to, and beyond, the target area, to attain a “crossability” of the pre-dilatation balloon (or other interventional device) that is superior to that of conventional balloon angioplasty catheters.

One of the objects of the subject invention is to use a highly flexible tapered elongated micro-catheter delivery tip to deliver a pre-dilatation balloon (or another interventional device) to, and beyond, a target lesion to be treated with angioplasty (or stenting) in a diseased human coronary artery.

It is an additional object of the present invention to provide a guide catheter extension/pre-dilatation system using an outer member (outer delivery sheath sub-system) and an inner member (interventional device delivery sub-system) fitted inside the outer sheath, both deliverable to, or beyond, the lesion area of treatment within a blood vessel where the inner member has a delivery tapered micro-catheter at its distal end with the pre-dilatation balloon member (or another interventional device) attached thereto which slides along a guidewire (prompted by pushing the sheath) in a substantially atraumatic fashion to, and beyond, a site of interest in a diseased coronary artery.

It is a further object of the present invention to provide a guide catheter extension sub-system (outer member) integrated with the pre-dilatation balloon (or another interventional device) sub-system (inner member), in which the outer member and the inner member can be coupled one to another to be integrally displaced along the guidewire to a lesion site. After the pre-dilatation procedure, the guide catheter extension sub-system (configured with an outer delivery sheath) may be advanced beyond the lesion, and the inner member (interventional device delivery sub-system) is withdrawn. The outer delivery sheath left in the guide catheter permits an easy deliverability of a stent (or other interventional device) to the lesion site inside the outer delivery sheath. The outer delivery sheath is then withdrawn exposing the stent (or other interventional device) to the lesion for definitive treatment.

It is an additional object of the present invention to provide a guide catheter extension/pre-dilatation system configured with an inner member which includes an inflation lumen (for inflation/deflation of the pre-dilatation balloon) which extends between an inflation port at the proximal end of the system in a surrounding relationship with the guidewire lumen formed within the inflation lumen and extending internally along the micro-catheter at the distal end of the inner member for passage of the guidewire therethrough.

It is another object of the present invention to provide a guide catheter extension/pre-dilatation system where the outer and inner members have tapered distal ends interfacing each other such that there is a virtually “seamless” transition between the inner and outer members which is beneficial for atraumatic passage of the system down a diseased artery.

Furthermore, it is an object of the present invention to provide the guide catheter extension/pre-dilatation system equipped with a “locking mechanism” between the inner member and the proximal portion of the outer member (outer sheath) operating to provide the integral passage of both the inner and outer members as a single unit for convenient deliverability of the pre-dilatation balloon and the outer sheath to, and beyond, the treatment site.

It is a further object of the present invention to provide a guide extension system configured with the pre-dilatation balloon (or other interventional device) delivery catheter deliverable to the treatment site inside a vascular structure in an atraumatic manner to attain easy passage of the balloon (or other interventional device) and the guide extension system therethrough, thus expediting the cardiac procedure and permitting percutaneous coronary intervention to be performed with less radiation dose and with virtually no risk of stent embolization, or drug loss with drug-eluting stents, from the stent delivery system.

It is another object of the present invention to provide a guide catheter extension/pre-dilatation system in which the outer tubular delivery sheath (which constitutes the guide catheter extension sub-system) may be formed from (or reinforced with) a flat wire helical coil (with a wire thickness of approximately 1 mil to 3 mils), which is either embedded in the plastic wall of the sheath, or has a very thin coating of plastic placed onto its inner and outer surfaces. This design reduces the wall thickness of the outer delivery tubular sheath to less than 7 mils, and, preferably, to around 5 mils. The micro-catheter in proximity to the pre-dilatation balloon (or another interventional device) positioned at the distal end of the inner member (also referred to herein as an interventional device delivery sub-system) is also envisioned as being formed from (or reinforced with) the flat wire helical coil, which may have a pitch changing along the micro-catheter length to provide a flexibility gradient beneficial for operation and atraumatic qualities of the subject system. Such a novel construction reduces the outside diameter of the subject system compared to existing guide extension systems.

Furthermore, it is an object of the present invention to provide a guide extension system having a shaft which employs a thin-walled, flat wire helical coil fabricated from a shape memory alloy such as Nitinol to prevent the possibility of kinking of the tubular outer delivery shaft of the guide catheter extension.

Still another object of the invention is to provide a tapered micro-catheter delivery system that has a balloon (or other interventional device) secured at its proximal portion to permit the balloon expansion, after it has been advanced into the coronary artery and to, and beyond, an area of interest.

A further object of the subject invention is to provide an outer delivery sheath whose distal end is tapered, and can be stretched during the removal of the inner member, thus forming a nearly flush (smooth) outer surface at the point at which the inner member exits the outer member.

In one aspect, the present invention constitutes an intravascular delivery system for percutaneous coronary intervention which is built with a guide catheter extension sub-system integrated with an interventional device (for example, pre-dilatation balloon) delivery sub-system for controllable advancement internally of a guide catheter in a blood vessel of interest to, or beyond, a treatment site.

The subject system is built with proximal section, a distal section, and a middle section interconnected between the proximal and distal sections.

The subject guide catheter extension/interventional device delivery system comprises an outer member formed by a substantially cylindrically contoured elongated flexible sheath (outer delivery sheath) defining a sheath lumen having a proximal end and a distal end. The outer delivery sheath extends between the middle section and the distal section of the subject system.

The subject system further includes an inner member which constitutes the interventional device (such as, for example, a pre-dilatation balloon) delivery sub-system having an elongated body defining an internal channel extending along its longitudinal axis. The inner member extends internally along the sheath lumen in a controllable relationship with the outer delivery sheath.

The inner member has a tapered distal end configured with a tapered delivery micro-catheter having an elongated body of a predetermined length. The tapered delivery micro-catheter slides along the guide wire during the controlled displacement of the outer member jointly with the inner member (as a single unit) inside the guide catheter along the blood vessel.

A pre-dilatation balloon member (or other interventional device) is secured at the tapered distal end of the inner member in close proximity to the tapered micro-catheter and is displaced along the guide wire along with the tapered micro-catheter along with the outer delivery sheath.

When the interventional device is a pre-dilatation balloon member, it is coupled in a sealed fluid communication with a balloon inflation system through the internal channel of the inner member. The pre-dilatation balloon member can assume a deflated configuration and an inflated configuration, as required by the cardiac angioplasty procedure.

The subject system further comprises an interconnection mechanism disposed in an operative coupling with the inner and outer members and controllably actuated by a surgeon to operate the guide catheter extension/interventional device delivery system in an engaged or disengaged modes of operation. Additional (second) “locking” of the inner and outer units may be attained via a connection at the proximal end of the two units and outside the body, to further enhance the integral movements of the inner and outer units.

In the engaged mode of operation, the subject inner and outer members, locked one to another by the interconnection mechanism, are controllably advanced (as a single unit) inside the guide catheter to the lesion location. Once the lesion location has been dilated, the outer delivery sheath may be advanced across the lesion integral with the inner member (the pre-dilation balloon is deflated). Subsequently, the inner member is disengaged from the outer delivery sheath, by deactuating the interconnection mechanism(s) and removed from the outer delivery sheath.

Alternatively, after the lesion location has been pre-dilated, the inner member is disengaged from the outer delivery sheath and removed therefrom, while the outer delivery sheath is advanced over the deflated balloon and across the lesion. This approach may further enhance the ability of the distal end of the “sheath” to be safely passed across the lesion.

In addition, upon removal of the inner member, the outer delivery sheath may be left in a place proximal to the lesion after the pre-dilatation procedure has been performed.

In any case scenario, during the inner member retraction (removal) from the outer delivery sheath (away from the guide catheter), the pre-dilatation balloon member is in its deflated configuration.

It is of importance, that the distal end of the inner member is positioned at a predetermined location external to the distal end of the outer delivery sheath. The inner member is capable exclusively of the retraction (withdrawal) from the outer delivery sheath, but is prevented from forward displacement relative the distal end of the outer delivery sheath beyond the predetermined location, as supported by the configuration of the interconnection mechanism. Specifically, in the engaged mode of operation, the inner member is coupled to the outer delivery sheath, both inside the guiding catheter and outside the body, and can be displaced relative thereto neither forward nor backward. In the disengaged mode of operation, the configuration of the interconnection mechanism also prevents the forward motion of the inner member permitting only backward displacement with respect to the outer delivery sheath.

In the engaged mode of operation, the inner and outer members of the subject system are engaged for a controllable integral displacement along the guide wire, and in the disengaged mode of operation, the inner and outer members are disengaged for a controllable retractional displacement of the inner member from the outer delivery sheath for withdrawal from the guide catheter after the pre-dilatation procedure has been performed.

Preferably, the micro-catheter is formed of a flexible material having differential flexibility along its length. The flexibility of the micro-catheter increases towards its distal end, and the tip is generally of a slightly smaller and tapered dimension relative to the more proximal shat of the micro-catheter. In one embodiment, the micro-catheter may be configured with a flat wire helical coil extending along the predetermined length of the micro-catheter. The pitch of the flat wire helical coil may change along the length of the micro-catheter to increase the flexibility of the micro-catheter towards its distal end.

It is of importance that the micro-catheter is an elongated member having a predetermined length in a cm range, and can reach the length of 1-3 cm, or longer. A diameter of the micro-catheter's cross-section at its distal end does not exceed 0.016″ (˜1 mm), while at its proximal end it does not exceed 0.032″.

The pre-dilatation balloon member attached to the distal tip of the inner member in close proximity to the micro-catheter has a proximal portion with a cross-sectional diameter not exceeding 0.032″, and a distal portion having a cross-sectional diameter not exceeding 0.027″.

The inner member extends inside the outer member along its length. The inner member is configured with an inflation lumen extending between a balloon inflation hub (at the proximal portion of the subject system) and the proximal portion of the pre-dilatation balloon member to serve as a passage for the inflation air from/to an inflation system for inflation/deflation of the pre-dilatation balloon as necessary in pre-dilatation/stenting procedure(s). The balloon inflation system is provided in the subject system to support a controllable inflation/deflation of the pre-dilatation balloon member.

The inflation lumen in the inner member is formed by an inflation lumen hypotube (extending along the proximal section and a portion of the middle section of the subject system) and an inflation lumen distal shaft (extending along the length of the middle and distal sections of the subject system). At the middle section, the inflation lumen hypotube and the inflation lumen distal shaft are overlappingly connected to provide a fluidly sealed passage of the inflation air between the balloon inflation system and the pre-dilatation balloon member.

The outer member's delivery sheath, at its distal end, is configured with a tapered outer tip, while the inner member, at its distal end, is configured with a tapered distal tip. The tapered distal tip of the inner member interfaces, at its outer surface, with an inner surface of the tapered outer tip of the sheath. It is of a paramount importance that a dimensional transition between the outer diameter of the outer tip of the outer member's sheath and the outer diameter of the distal tip of the inner member does not exceed 0.004″ in order to form a substantially gradual (flush) transition therebetween and provide a “smooth” outer surface at the distal portion of the subject guide catheter extension/pre-dilatation system for atraumatic crossability of the subject system and for the integral displacement of the inner and outer members as a single unit.

The subject system further comprises an outer member pusher which is coupled, at its distal end, to the proximal end of the outer member's delivery sheath. The outer member pusher is actuated by a surgeon (operator) to control the integral displacement of the outer member along with the inner member (when engaged with the outer member) along the guide wire. The outer member pusher includes a proximal round wire pusher portion and a flattened distal portion at its distal end. It is not partially cylindrical.

The distal end of the outer member pusher may have a flattened arcuated configuration cooperating with a contour of the external surface of the inner member at its proximal end. The distal end of the outer member pusher is fixedly attached to the proximal end of the outer member's delivery sheath. The outer member pusher may have a pusher handle attached to the proximal end of the outer member pusher, which is held and manipulated by a surgeon.

The inner member also may be equipped with an inner member pusher, which may be configured as a wire welded (or glued) to the proximal end of the inner member, for example, in proximity to (or to) the inflation hub. An inner member pusher's handle may be attached at the proximal end of the inner member pusher. By manipulating the inner and/or outer member's pushers, a surgeon may control the withdrawal of the inner member away from the outer delivery sheath in the disengaged mode of operation upon the pre-dilatation procedure has been performed. The handles of the inner and outer members' pushers may be configured with a mechanism permitting a releasable locking of the inner and outer members one to another to enhance the integral cooperation thereof in the engaged mode of operation.

The internal channel formed in the inflation lumen distal shaft also serves for accommodating a guide wire lumen for passage of the guide wire between the RX port (formed in the wall of the inflation lumen distal shaft) and the distal end (including the tapered micro-catheter) of the inner member.

The interconnection (engagement/disengagement) mechanism in the subject system is envisioned in a number of alternative embodiments, each of which however has a common feature, which is the prevention of the forward displacement of the inner member relative to the outer delivery sheath. The interconnection mechanism is configured in a fashion to permit the integral motion of the inner and outer member (as a single unit) in the engaged mode of operation, and exclusively a backward displacement of the inner member relative to the outer delivery sheath in the disengaged mode of operation for withdrawal of the inner member therefrom after the pre-dilatation has been performed.

For example, in one embodiment, the interconnection mechanism may be configured as a friction-based mechanism which is tapered at its proximal end so that its diameter I the proximal end is larger than the diameter of the cooperating portion of the outer member for preventing the forward movement of the inner member respective to the outer member. Only a backward movement of the inner member relative to the outer delivery sheath is permitted in the subject system.

In another embodiment, the interconnection mechanism may include at least one engagement button extending above an external surface of the inner member, and at least one engagement channel configured at the proximal end of the sheath of the outer member. The engagement button may be releasably engaged (by operating the outer and inner members' pushers) in the engagement channel for locking the inner and outer member one to another.

In an alternative embodiment, the subject interconnection mechanism includes a snap-fit mechanism in various configurations.

Preferably, the subject system is envisioned to be configured with a flat wire helical coil member forming at least a portion of walls of the outer member's sheath and/or the inner member's micro-catheter. The flat wire helical coil which may be embedded in the walls of the sheath and/or micro-catheter, may be formed of a radio-opaque material, preferably including a shape memory alloy, such as Nitinol.

It is envisioned that radio-opaque markers are attached to the distal ends of the sheath and the micro-catheter, as well as at the proximal and distal portions of the pre-dilatation balloon member, to facilitate a surgeon in performing the cardiac procedure.

In another aspect, the present invention constitutes a method for intravascular treatment using a guide catheter extension sub-system integrated with the interventional device delivery sub-system (for example, pre-dilatation sub-system) in cooperation with a guide wire and guide catheter. The subject method comprises the steps of:
assembling a guide catheter extension/pre-dilatation system having:
   an outer member formed by a flexible substantially cylindrically contoured elongated outer delivery sheath defining a sheath lumen having a proximal end and a distal end,
   an inner member having an elongated body defining an internal channel extending along its longitudinal axis. The inner member has a distal end configured with a tapered delivery micro-catheter having an elongated body of a predetermined length (preferably, in cm range), and an interventional device (such as, for example, a pre-dilatation balloon member) secured at the distal end of the inner member in close proximity to the proximal section of the micro-catheter. The inner member is extended inside the sheath lumen in a controllable relationship with the outer delivery sheath, and
   an interconnection mechanism disposed in an operative coupling with the inner and outer members of the guide catheter extension/pre-dilatation system and controllably actuated (by a surgeon) to operate the guide catheter extension system in an engaged or disengaged modes of operation.

In the engaged mode of operation, the inner and outer members of the subject system are engaged for a controllable integral displacement along the guide wire inside the guide catheter. In the disengaged mode of operation, the inner and outer members are disengaged for a controllable retractional displacement of the inner member away from the outer delivery sheath after the pre-dilatation procedure has been performed.

In the engaged mode of operation, the subject inner and outer members, locked one to another by the interconnection mechanism, are controllably advanced (as a single unit) inside the guide catheter to the lesion location. Once the lesion location has been dilated, the outer delivery sheath may be advanced across the lesion integral with the inner member (the pre-dilation balloon is deflated). Subsequently, the inner member is disengaged from the outer delivery sheath (by deactuating the interconnection mechanism) and removed from the outer delivery sheath.

Alternatively, upon the lesion location has been pre-dilated, the inner member is disengaged from the outer delivery sheath and removed therefrom, while the outer delivery sheath is advanced across the lesion.

In addition, upon removal of the inner member, the outer delivery sheath may be left in a place proximal to the lesion after the pre-dilatation procedure has been performed, and a stent may be delivered over the same distally placed guidewire, inside the outer delivery sheath.

In any case scenario, during the inner member retraction (removal) from the outer delivery sheath (away from the guide catheter), the pre-dilatation balloon member is in its deflated configuration.

It is of importance, that the inner member is prevented from distal (forward) displacement relative the distal end of the outer delivery sheath (a) by locking thereto in the engaged mode of operation, and (b) in the disengaged mode of operation, exclusively the backward motion of the inner member is supported by a specific configuration of the interconnection mechanism.

In the engaged mode of operation, the inner and outer members of the subject system are engaged for a controllable integral displacement along the guide wire, and in the disengaged mode of operation, the inner and outer members are disengaged for a controllable retractional displacement of the inner member from the outer delivery sheath for withdrawal from the guide catheter after the pre-dilatation procedure has been performed.

The subject method further includes the following steps:
upon assembling the guide catheter extension/pre-dilatation system, extending a guide wire along the internal channel of the inner member with a proximal end of the guide wire extending beyond an RX port formed in the wall of the inner member, and a distal end of the guide wire extending beyond a distal end of the delivery micro-catheter;
advancing the distal end of the guide wire into a blood vessel of interest towards, and beyond, a treatment site;
controlling the interconnection mechanism to establish the engaged mode of operation;
in the engaged operational mode, advancing the guide catheter extension sub-system integral with the pre-dilatation sub-system as a single unit (with the pre-dilatation balloon member in its deflated configuration), within the guide catheter along the blood vessel of interest, with the micro-catheter sliding along the guide wire towards the treatment site until the balloon member is brought substantially in alignment with the treatment site.

Subsequent to bringing the pre-dilatation balloon member to a desired position, the subject method continues with inflation of the balloon member (using the inflation system) to press down the plaque formation and widen the blood passage in the blood vessel at the treatment site.

After the pre-dilatation procedure has been performed, the balloon member is deflated, and the guide catheter extension/pre-dilatation system is advanced beyond the treatment site. The inner member is disengaged from the outer delivery sheath, and is removed away from the blood vessel along the guide wire to permit introduction of another balloon catheter with a stent secured to the balloon (or other interventional device) for the stenting procedure. The balloon/stent catheter is advanced to the treatment site over the guidewire and inside the outer delivery sheath. The outer delivery sheath is withdrawn (“un-sheathing”) subsequent to exposing the stent and its delivery balloon at the treatment site. The stent balloon is then inflated, and the stent is deployed.

These and other objects and advantages of this invention will become apparent to a person of ordinary skill in this art upon reading the detailed description of the subject invention in conjunction with the Patent Drawings.

### BRIEF DESCRIPTION OF THE DRAWINGS

FIGS. 1A-1C schematically depict the conventional stent angioplasty procedure;

FIGS. 2A-2B show schematically the conventional over-the-wire balloon catheter (FIG. 2A) and Rapid Exchange (RX) balloon catheter (FIG. 2B);

FIG. 3 shows schematically the subject guide catheter extension/pre-dilatation system advanced to the target site within a coronary artery;

FIGS. 4A-4C show schematically the subject guide catheter extension/pre-dilatation system, where FIG. 4A shows the assembled inner and outer members, FIG. 4B shows the inner member, and FIG. 4C details the middle section of the subject system;

FIG. 5 is representative of the inflation hub at the proximal section of the subject system;

FIG. 6A-6D are representative of a middle section of the subject inner member with FIG. 6A showing a longitudinal section of the inflation lumen hypotube interconnected with the inflation lumen distal shaft in the inner member, FIG. 6B detailing a longitudinal section of the skived portion of the inflation lumen hypotube, FIG. 6C showing a longitudinal section of the inner member depicting an RX guide wire (GW) port formed in the inflation lumen distal shaft, and FIG. 6D showing an isometric view of the RX port portion of the inner member shown in FIG. 6C;

FIG. 7 shows a longitudinal section of the inner member detailing the distal end of the inflation hypotube at the junction with the inflation lumen distal shaft;

FIGS. 8A-8C show the distal section of the subject system with FIG. 8A presenting the inflated balloon member, FIG. 8B presenting the deflated balloon member, and FIG. 8C detailing the inflation lumen/balloon junction;

FIGS. 9A-9B depict the longitudinal section of the distal section of the subject inner member detailing the balloon's 3 mm distal and proximal tapers (FIG. 9A) and the balloon's 6 mm distal and proximal tapers (FIG. 9B);

FIGS. 10A-10B depict the distal section of the subject inner member with the inflated balloon (FIG. 10A) and deflated balloon (FIG. 10B);

FIGS. 11A-11B are representative of the alternative implementation of the subject system with a “chocolate” type balloon with FIG. 11A showing a full (inner/outer members) catheter assembly, and FIG. 11B showing the subject inner member sub-assembly;

FIGS. 12A-12B depict a side view (FIG. 12A) and an isometric view (FIG. 12B) of the proximal portion of the subject outer member;

FIGS. 13A-13B depict a side view (FIG. 13A) and an isometric view (FIG. 13B) of the proximal portion of the subject outer member in its alternative embodiment;

FIGS. 14A-14B depict a side view (FIG. 14A) and an isometric view (FIG. 14B) of another alternative embodiment of the proximal portion of the subject outer member;

FIGS. 15A-15E depict a friction lock ring embodiment of the subject interconnection mechanism with FIG. 15A showing the outer member coupler sub-assembly, FIG. 15B showing the inner member cooperating sub-assembly, and FIGS. 15C, 15D, 15E showing the side, top, and isometric views of the lock ring interconnection mechanism, respectively;

FIGS. 16A-16D depict an alternative snap-fit “Omega-Shape” embodiment of the subject interconnection mechanism, with FIGS. 16A-16B showing the outer and inner member sub-assemblies, respectively, and FIGS. 16C-16D being a top and isometric views, respectively, of the interconnection mechanism;

FIGS. 17A-17D depict another alternative snap-fit “Simple Rib” embodiment of the subject interconnection mechanism with FIGS. 17A-17B detailing the outer and inner member sub-assemblies, respectively, and FIGS. 17C-17D being the top and isometric views, respectively, of the subject interconnection mechanism;

FIGS. 18A-18D depict another alternative snap-fit “W-Shape” embodiment of the subject interconnection mechanism, with FIGS. 18A-18B detailing the outer and inner member sub-assemblies, respectively, and FIGS. 18C-18D being the top and the isometric view, respectively, of the subject interconnection mechanism;

FIGS. 19A-19D depict an alternative circumferential snap-fit embodiment of the subject interconnection mechanism, with FIGS. 19A-19B detailing the outer and inner member sub-assemblies, respectively, and FIGS. 19C-19D being the top and isometric views, respectively, of the subject interconnection mechanism;

FIGS. 20A-20C depict another alternative 3 post snap-fit embodiment of the subject interconnection mechanism with FIG. 20A being a top view and FIGS. 20B-20C being side isometric views, respectively, of the subject interconnection mechanism;

FIGS. 21A-21B depict the top and isometric views, respectively, of the 3 post snap-fit embodiment of the interconnection mechanism with 90° angular spacing between the posts, where FIG. 21B details an isometric cross-section of FIG. 21A taken along lines A-A;

FIGS. 22A-22B depict the top and isometric view of an alternative 3 post snap-fit interconnection mechanism, respectively, where FIG. 22B shows an isometric cross-section of FIG. 22A taken along lines A-A; and

FIGS. 23A-23F illustrate schematically a sequence of steps during the cardiac intervention procedure using the subject guide catheter extension/pre-dilatation system.

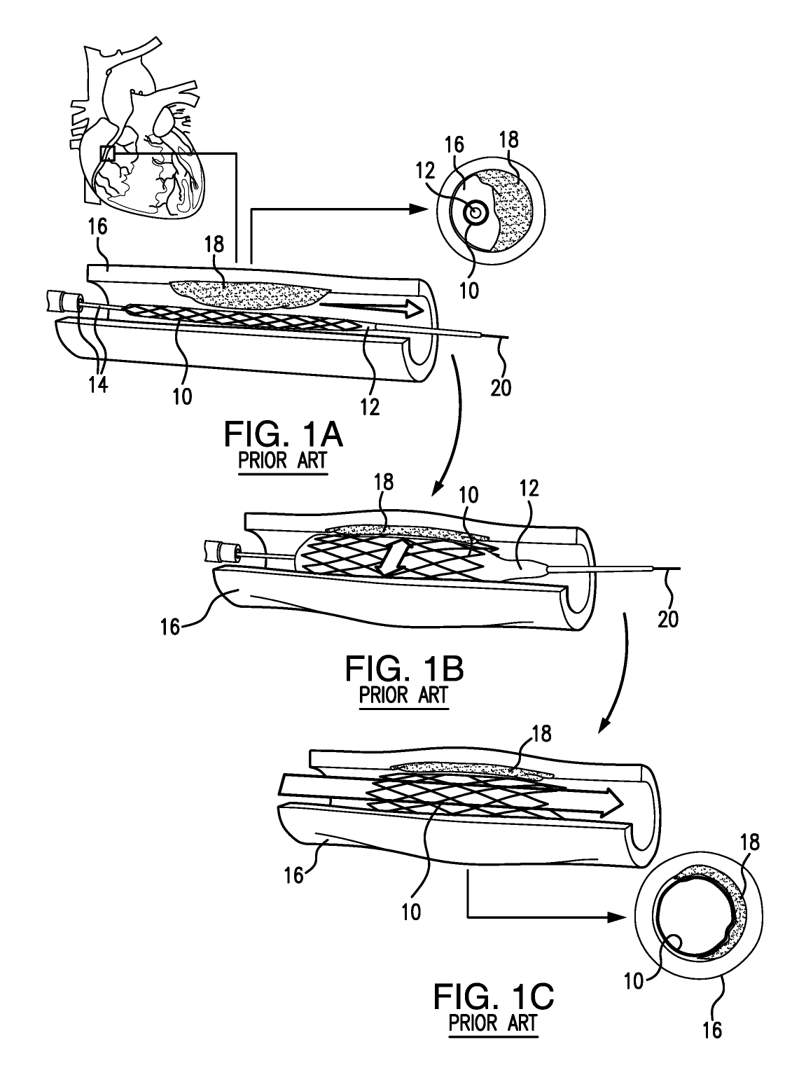

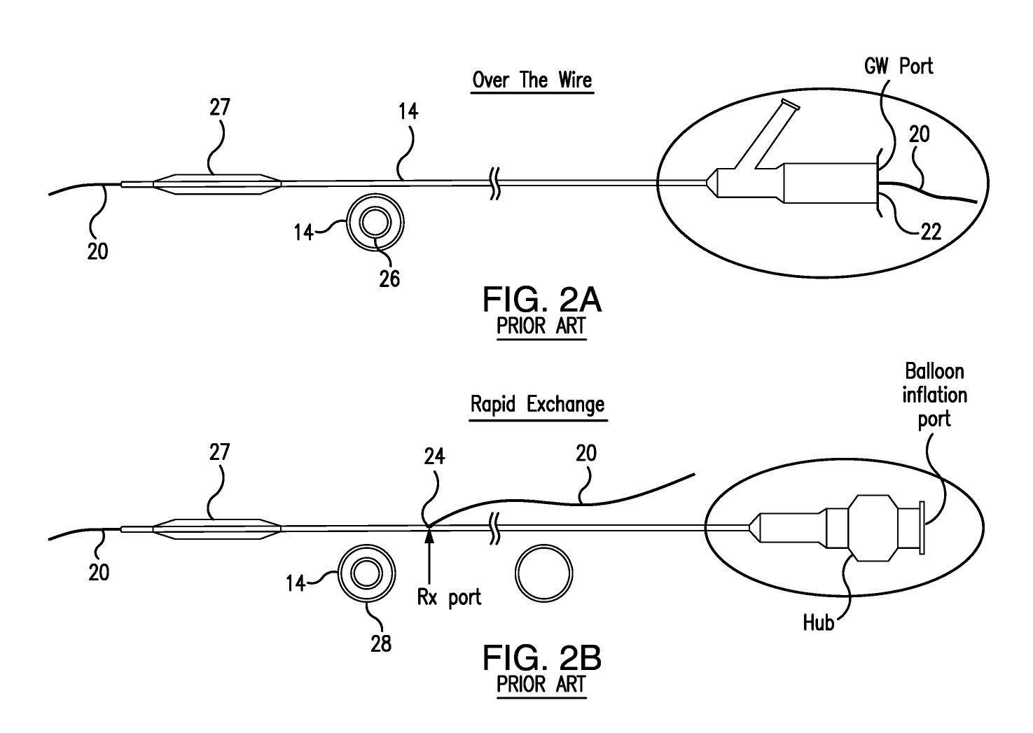

### DETAILED DESCRIPTION OF THE PREFERRED EMBODIMENTS

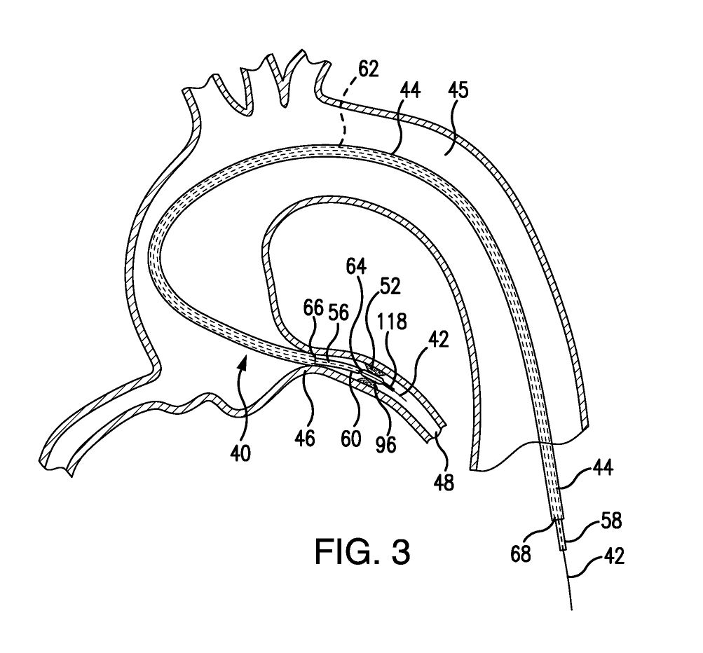

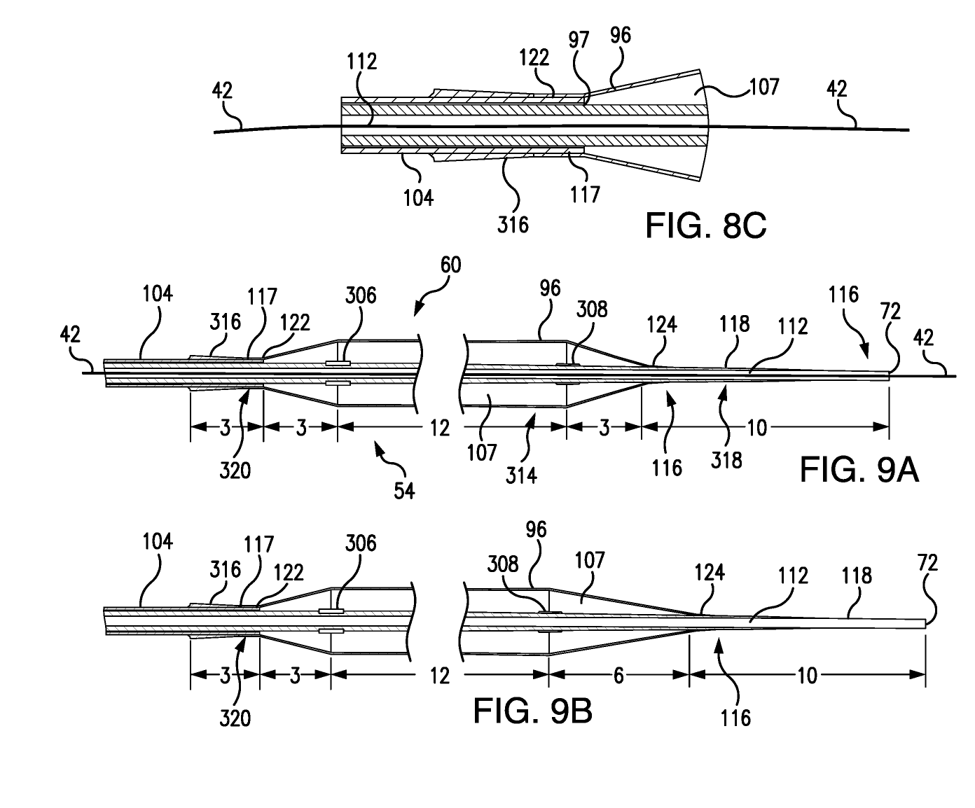

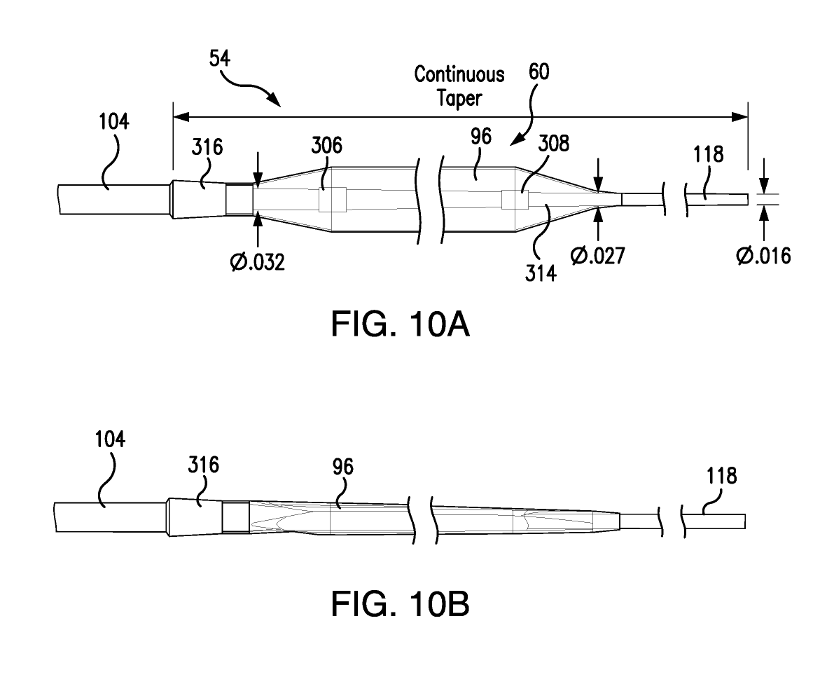

Depicted in FIGS. 3-23F, is the subject intravascular delivery system 40 and method for percutaneous coronary intervention. The subject system 40 includes a guide catheter extension sub-system (outer member) and an interventional device delivery sub-system (inner member) cooperating under control of a surgeon during a cardiac procedure. Although the interventional device delivery sub-system may be used for delivery of various cardiac interventional devices, in one of implementations, as an example only, but not to limit the scope of the subject invention to this particular embodiment, the subject interventional device delivery sub-system will be further described as adapted for delivery of a balloon member for performing the pre-dilatation procedure.

Therefore, in the exemplary embodiment described herein, the subject system 40 is referred to herein as a guide catheter extension/pre-dilatation system which is used for cardiac procedures in conjunction with a guide wire 42 and a guide catheter 44. As shown in FIG. 3, at the initial stage of the cardiac procedure, the guidewire (GW) 42 extends into the blood vessel 45, and the guide catheter 44 is advanced through the blood vessel 45 (such as the aorta) along the guide wire 42 to a position adjacent to the ostium 46 of the coronary artery 48. The guidewire 42 is used during the cardiac procedure to guide the guide catheter 44 and subsequently the subject guide catheter extension/pre-dilatation system 40 (inside the guide catheter 44) within the artery 48 toward a target location 52, as will be detailed in following paragraphs.

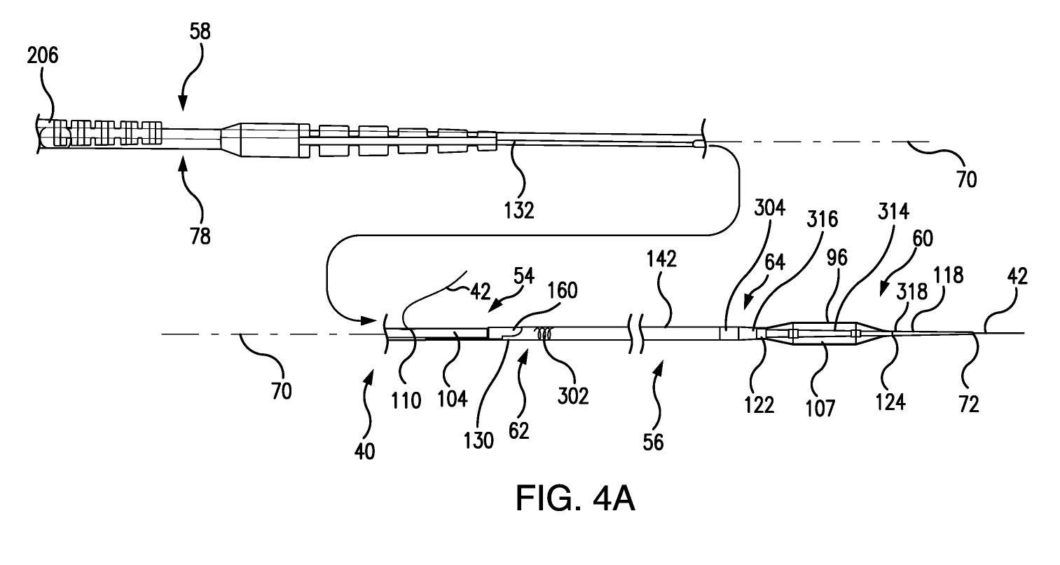

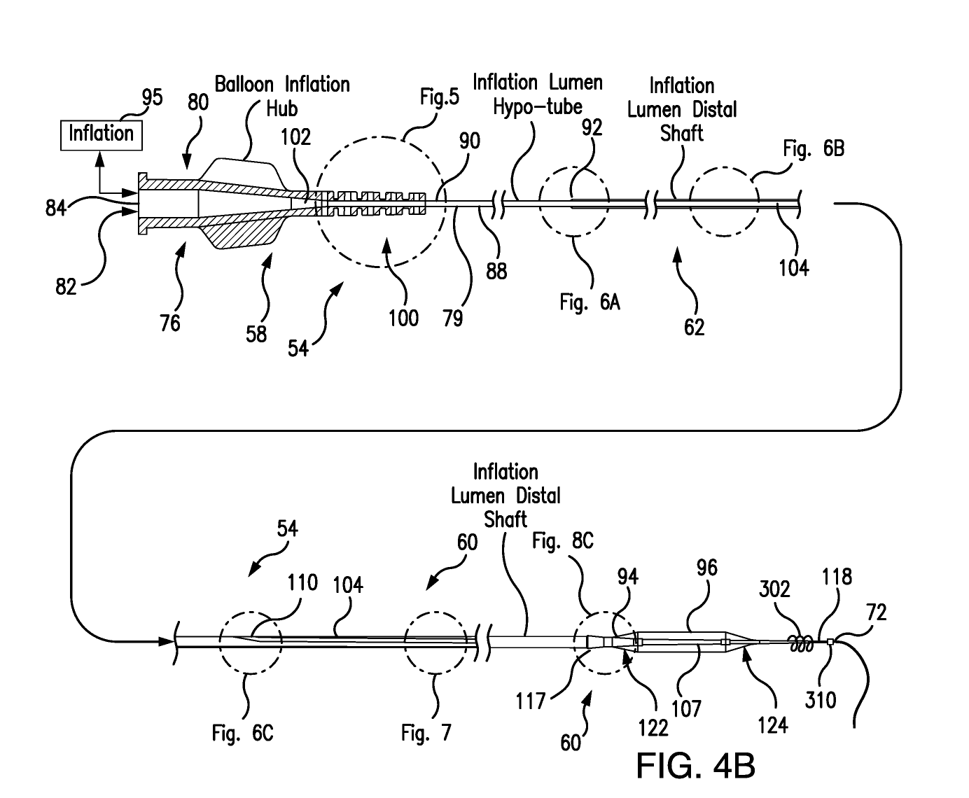

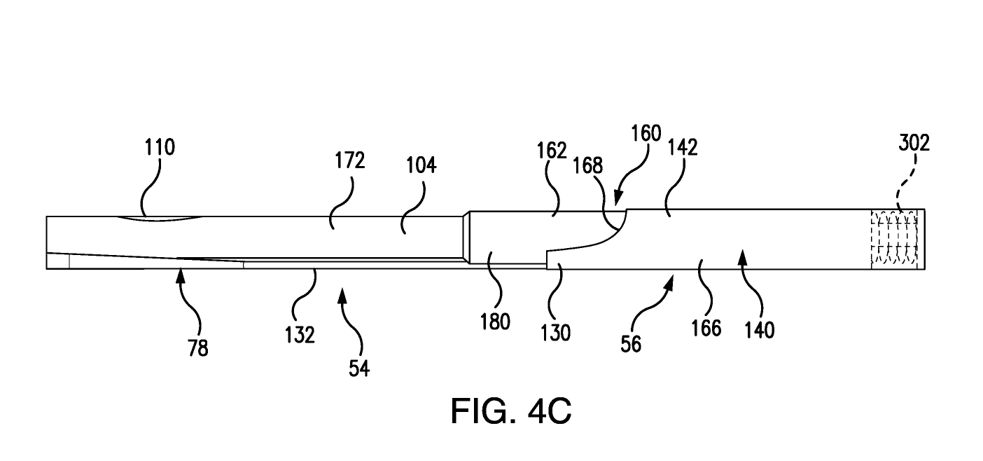

As shown in FIGS. 4A-4C, the subject guide catheter extension/pre-dilatation system 40 includes a balloon catheter sub-system 54 (also referred to herein as an inner member or a pre-dilatation sub-assembly) and a guide catheter extension sub-system 56 (also referred to herein as an outer member). The inner member 54 interacts with the outer member 56 and can be engaged with or disengaged from the outer member 56, as required by the cardiac procedure.

The subject system 40 includes a proximal section 58, a distal section 60, and a middle section 62 extending between and interconnecting the proximal and distal sections 58, 60. A pre-dilatation balloon member 96 is carried at the distal section 60 of the inner member 54. The distal section 60 of the inner member 54 also is configured with an elongated tapered micro-catheter 118, as will be detailed in the following paragraphs.

The subject guide extension/pre-dilatation system 40 is shown in FIG. 3 being extended within a lumen (internal channel) 68 of the guide catheter 44. In order to reliably reach the target location 52, and in some cases, pass beyond the target location 52, the subject guide extension/pre-dilatation system 40, as shown in FIG. 3, is advanced through the guide catheter 44 beyond a distal end 66 of the guide catheter 44 deep into the coronary artery 48. The subject system 40, by extending beyond the distal end 66 of the guide catheter 44, provides an adequate reachability for the pre-dilatation balloon 96 to the target location 52, and, by extending beyond the ostium 46 of the coronary artery 48, stabilizes the positioning of the guide catheter 44 and allows for an improved accessibility for the subject system 40 into the coronary artery 48 and to the target site 52.

As shown in FIGS. 3, 4A-4B, 6C-6D, 7, 8A-8C, 9A, and 23A-23F, the guide wire 42 extends internal the guide catheter extension/pre-dilatation system 40, and exits the system 40 with the distal end of the GW 42 beyond the outermost end 72 of the distal section 60 and with the proximal end of the GW 42 at the middle section 62 in a manner detailed in further paragraphs.

In operation, the inner member 54 and the outer member 56 coupled one to another are advanced (as a single unit) along the guide wire 42 inside the guide catheter 44 positioned within the blood vessel 45, and extend beyond the distal end 66 of the guide catheter 44 to reach the target lesion site 52. Once the subject balloon catheter sub-system (inner member) 54 reaches the lesion site 52, and the balloon member 96 is positioned in alignment with the lesion site 52, the intended pre-dilatation procedure may be performed. Once the pre-dilatation has been performed, the outer member 56 may be advanced across the lesion as an integral unit with the inner member 54, with subsequent disengagement of the inner member 54 from the outer member 56 for withdrawal of the inner member from the outer member.

Alternatively, after the pre-dilatation procedure has been performed, the inner member 54 may be disengaged from the outer member 56, while the outer member 56 is advanced across the dilated lesion. In addition, the outer member 56 may be left in proximity to the lesion after the pre-dilatation has been performed and the inner member 54 has been removed.

In any case scenario, the outer member 56 remaining in proximity to the pre-dilated lesion may be used for delivery of a stent inside the outer member 56 to the lesion site. The outer member 56 is removed from the guide catheter 44 once the stent is installed (deployed) at the lesion site.

As will be presented in further paragraphs, a care is taken in the subject system to prevent the inner member 54 from forward displacement inside the outer member 56. Exclusively a backward displacement of the inner member 54 relative to the outer member 56 is permitted to support retraction of the inner member from the outer member subsequent to the pre-dilatation of the lesion.

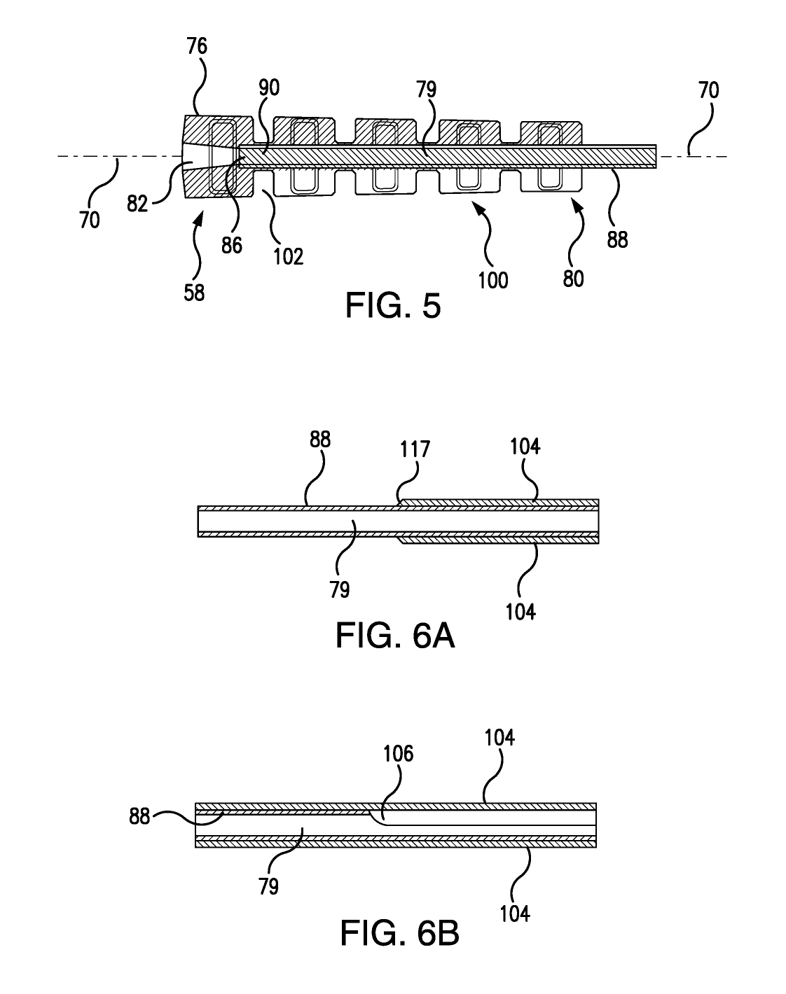

Referring to FIGS. 4A-4C, the proximal section 58 of the subject guide extension/pre-dilatation system 40 is represented by a balloon inflation hub 76 of the inner member 54 (best depicted in FIGS. 4B and 5) and a proximal end 78 of an outer member 56 (also shown in FIGS. 4C, 12A-12B, 13A-13B, and 14A-14B).

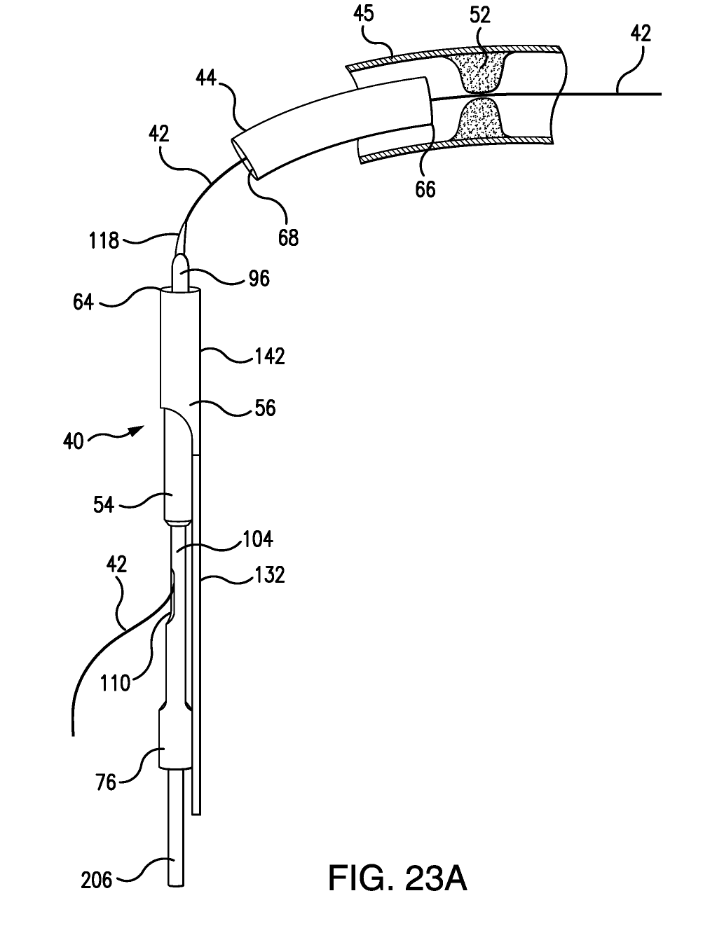

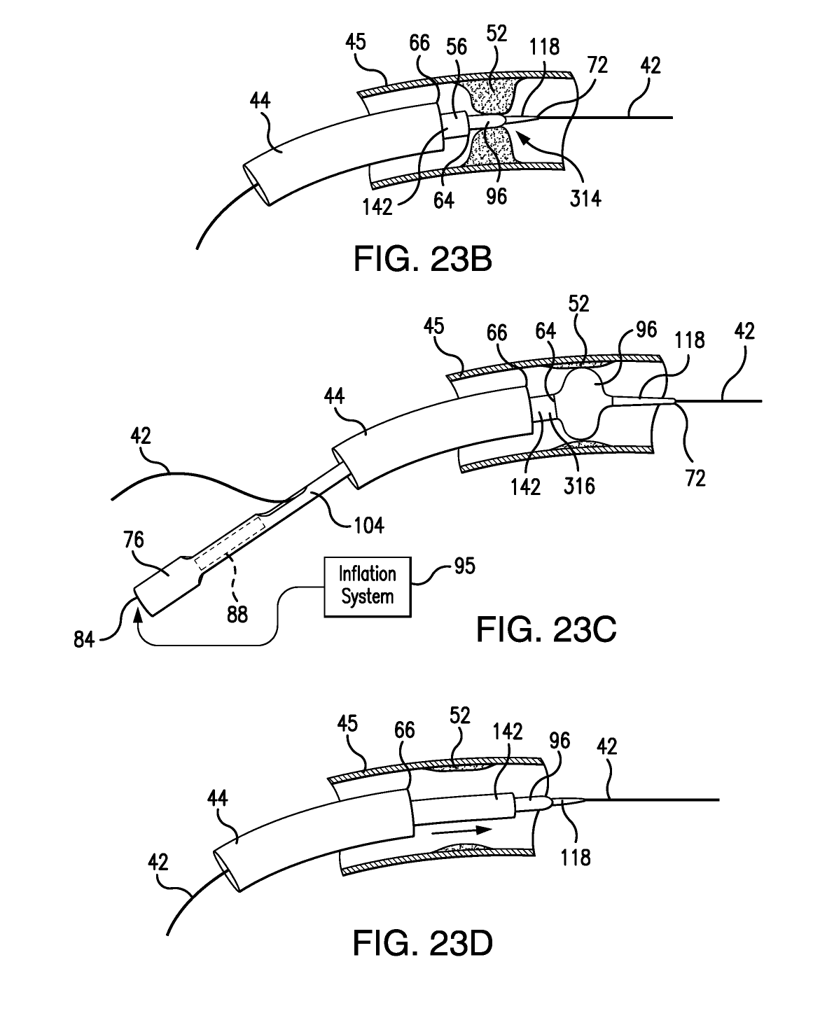

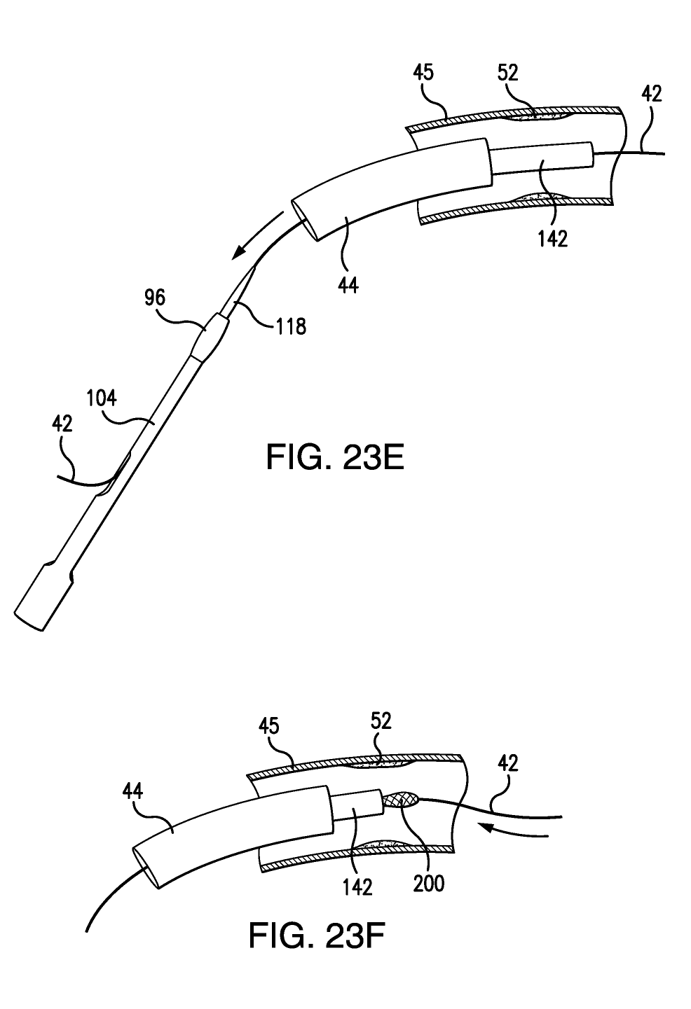

Referring to FIGS. 4B, 5, and 6A-6D, 7 and 8C, the inner member (also referred to herein intermittently as the balloon catheter sub-system or pre-dilatation balloon delivery sub-system) 54 is configured with an internal inflation channel 79 extending between the inflation hub 76 and the pre-dilatation balloon member 96. The internal inflation channel 79 serves as a passage for inflation air between a balloon inflation system 95 (shown in FIGS. 4B and 23C) and the balloon member 96 for the controlled inflation/deflation of the balloon member 96 as prescribed by the cardiac procedure.

The internal inflation channel 79 is formed by an inflation lumen hypotube 88 and an inflation lumen distal shaft 104 overlappingly interconnected each to the other in a fluidly sealed manner to be further detailed in following paragraphs.

The inflation hub 76 located at the proximal end 80 of the inner member 54 is configured with an internal cone-shaped channel 82 which is connected by its proximal opening 84 to the balloon inflation system 95 (schematically shown in FIGS. 4B and 23D).

The balloon inflation system 95 may be a manual or an automatic system. In preferred automatic embodiment, the balloon inflation system 95 includes an electronic sub-system, a pneumatic sub-system and control software with a corresponding user interface. The electronic sub-system, under control of the control software, supplies power to solenoid pressure valves (which are fluidly coupled to the balloon inflation hub 76) to control the pressurizing/depressurizing of the balloon member 96 with fluid or air flow.

As shown in FIGS. 4B and 5, the internal cone-shaped channel 82 of the balloon inflation hub 76 is configured with a distal opening 86 which is coupled to the inflation lumen hypo-tube 88. The proximal end 90 of the inflation lumen hypo-tube 88 is coupled to the distal opening 86 of the internal cone-shaped channel 82 of the balloon inflation hub 76 in a fluidly sealed fashion to support passage of the inflation air between the balloon member 96 at the inflation system 95.

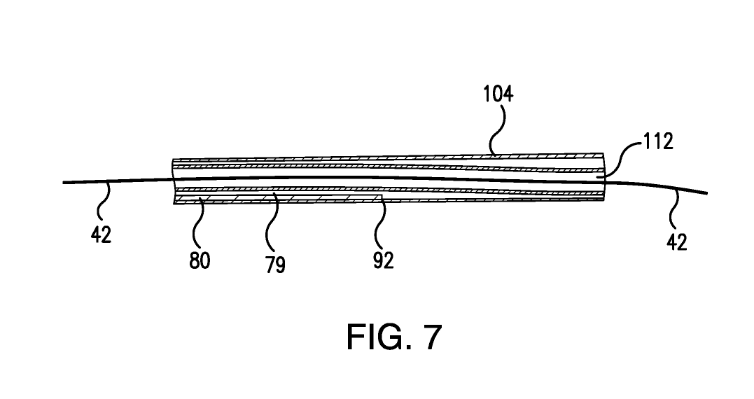

The inflation lumen hypo-tube 88 extends through the length of the proximal section 58 and a portion of the middle section 62 of the subject system 40 and terminates with its distal end 92 at the distal section 60, as shown in FIGS. 4B and 7.

As shown in FIGS. 4A-4B and 5, a flexible serrated member 100 is provided at the proximal end 90 of the inflation lumen hypo-tube 88 which is coupled to the distal end 102 of the balloon inflation hub 76. The serrated flexible member 100 supports the proximal end 90 of the inflation lumen hypo-tube 88 and provides a flexible bending of the structure when manipulated by a surgeon.

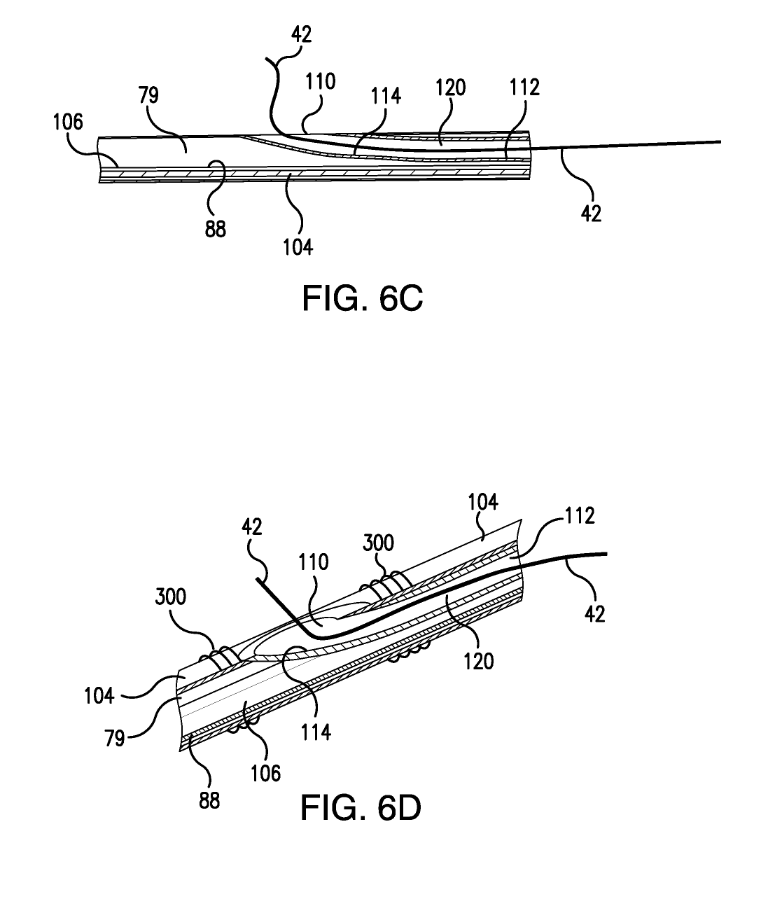

As shown in FIGS. 4A-4C, 6A-6D, 7 and 8C, the inflation lumen distal shaft 104 extends between the proximal section 58 along the middle section 62 and ends at the distal section 60. FIG. 6A details the junction between the inflation lumen hypo-tube 88 and the inflation lumen distal shaft 104. The inflation lumen hypo-tube 88 does not extend all the way through the inner member 54 but terminates at its distal end 92 (as shown in FIGS. 4B and 7).

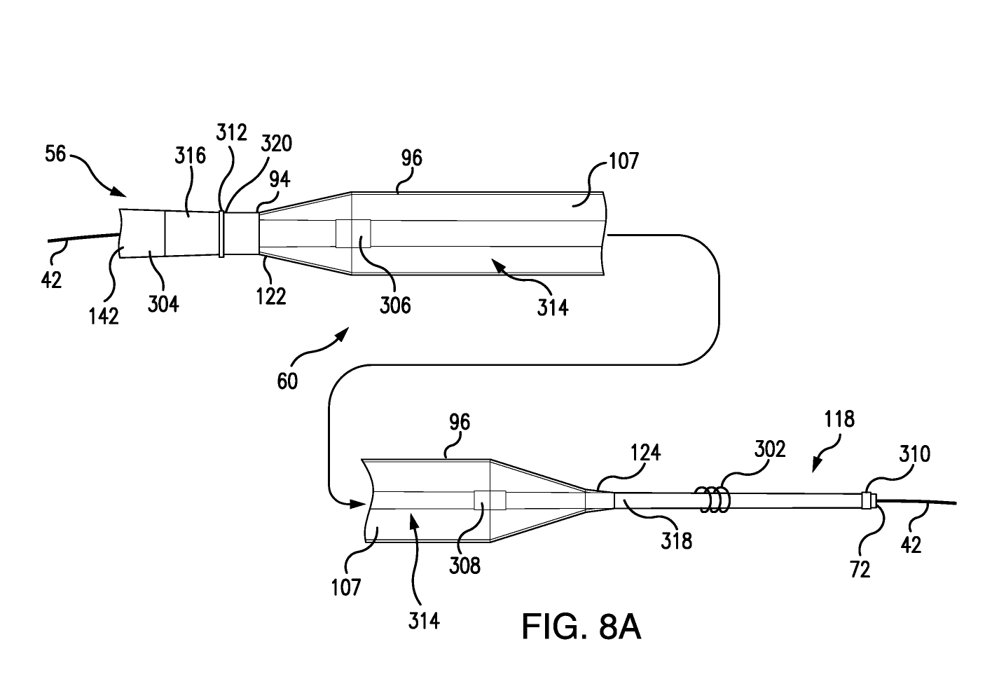

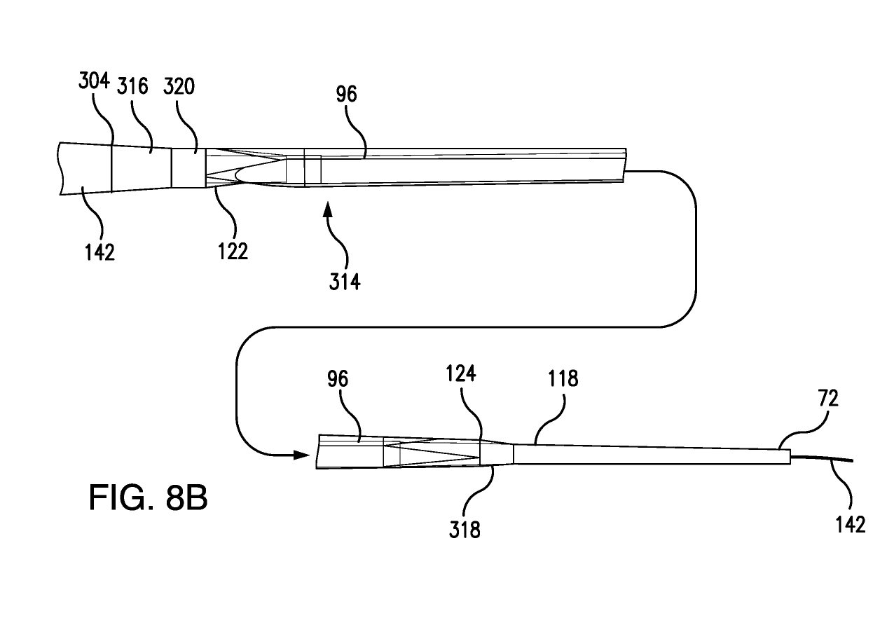

Referring to FIGS. 6B-6D, the inflation lumen hypo-tube 88 has a skived distal portion 106 which is coaxially enveloped by the wall of the inflation lumen distal shaft 104 so that the inflation lumen hypo-tube 88, in conjunction with the inflation lumen distal shaft 104, provide a sealed fluid communication between the balloon inflation system 95 and the internal chamber 107 of the balloon member 96, as shown in FIGS. 8A-8C, for controlled inflation/deflation of the balloon member 96 as required by the cardiac procedure.

FIGS. 4B and 6C-6D illustrate that the inflation lumen distal shaft 104 is configured with a rapid exchange (RX) guidewire (GW) port 110 at which a GW lumen 112 begins with its proximal end 114. The GW lumen 112 extends between the RX GW port 110 inside the inflation lumen distal shaft 104 through the entire length of the distal section 60 of the inner member 54. The GW lumen 112 forms an internal channel with the proximal end 114 corresponding to the RX GW port 110 and a distal end 116 corresponding to the outermost distal end 72 of the distal section 60 of the inner member 54. As shown in FIGS. 9A-9B, at the distal section 60, the GW lumen 112 extends beyond the distal end 117 of the inflation lumen distal shaft 104. The distal end 116 of the GW lumen 112 constitutes a gradually tapered portion 118 which is referred to herein as a delivery micro-catheter.

Referring to FIGS. 4A-4B, 8A-8C, 9A-9B, 10A-10B, and 11A-11B, the inner member (balloon catheter sub-system) 54 is configured with a tapered distal tip 314 at the distal section 60. The tapered distal tip 314 is equipped with the pre-dilatation balloon member 96 which is secured onto the tapered distal tip 314 in close proximity to the micro-catheter 118. The pre-dilatation balloon member 96 is secured to the inner member's tapered distal tip 314 for supporting the pre-dilatation/stenting procedure, as required for the cardiac treatment of a patient.

The balloon member 96 has a proximal portion 122 and a distal portion 124. The balloon member 96 is attached (secured) at the distal section 60 in proximity to the delivery micro-catheter 118 with its proximal portion 122 coupled to the distal end 117 of the inflation lumen distal shaft 104, and with the distal portion 124 of the balloon 96 to the outer surface of the micro-catheter 118.

The balloon member 96 may intermittently assume a deflated (folded) and an inflated (expanded) configurations. The deflated (folded) configuration is used during insertion and/or withdrawal of the subject system relative to the blood vessel. The the balloon is inflated (expanded) when in place (at the target site 52) to widen the blood vessel and compress the plaque for pre-dilatation procedure, or for the stenting procedure (when a stent is delivered to the treatment site on a balloon). When inflated, the balloon 96 assumes the inflated/open configuration shown in FIGS. 4A-4B, 8A, 8C, 9A-9B, 10A, 11A-11B and 23C for pre-dilatation of the diseased blood vessel. When deflated, the balloon member 96 assumes the deflated configuration shown in FIGS. 8B, 10B, 23B, and 23D-23E.

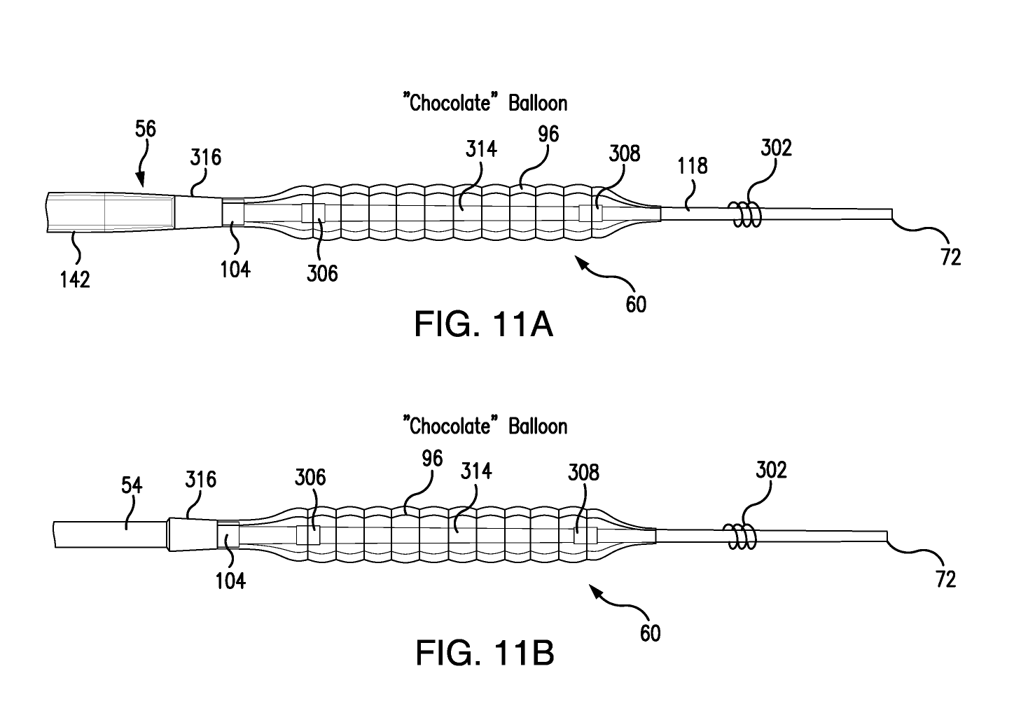

FIGS. 4A-4B, 8A-8B, 9A-9B, and 10A-10B show the balloon 96 having a smooth surface, while as shown in FIGS. 11A-11B, the balloon may have a “chocolate” configuration. The “chocolate” balloon catheter is an over-the-wire balloon dilatation catheter with a braided shaft and an atraumatic tapered tip. The balloon, when expanded, is constrained by a nitinol structure that creates small “pillows” and grooves in the balloon.

Referring now to FIGS. 4A, 4C, 8A-8C, 11A and 12A-12B, 13A-13B, 14A-14B, the outer member (also called the guide catheter extension sub-system) 56 is formed with a cylindrical outer delivery sheath 142 having an internal channel 144 extending internally therealong. A coupler 140 is formed at the proximal end 130 of the cylindrical sheath 142 in encircling relationship therewith.

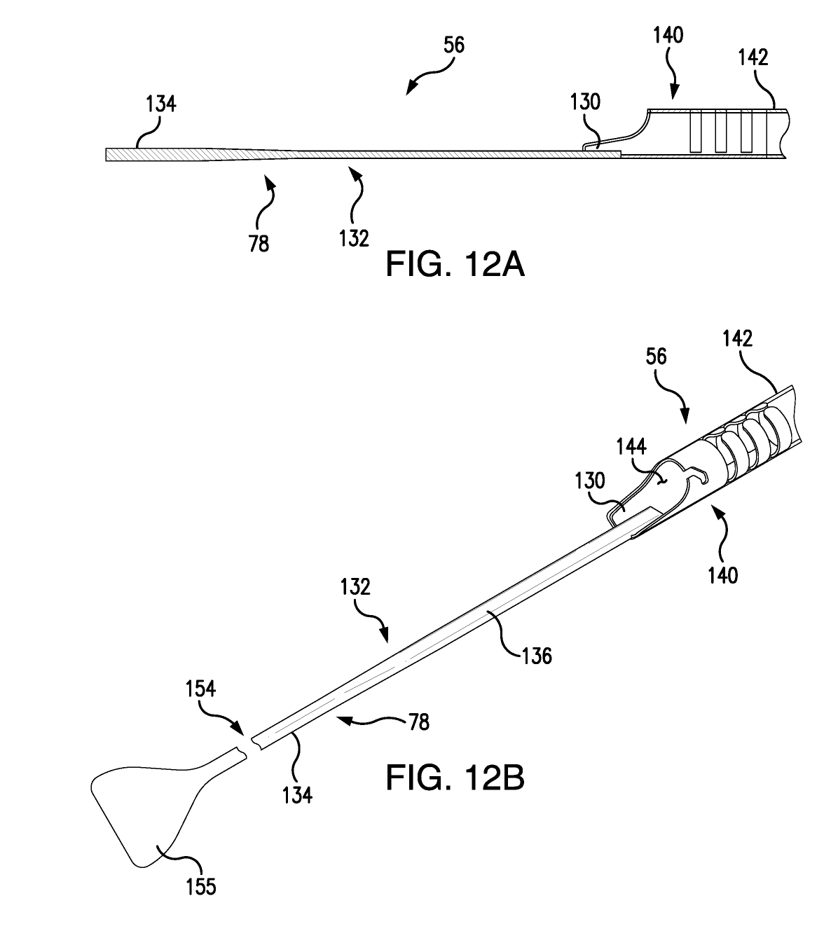

At the proximal end 78, the outer member 56 includes an outer member pusher 132, which, as shown in FIG. 12A-12B, in one embodiment, is a solid wire which may have a round wire proximal section 134, and a flattened distal portion 136 which is welded or otherwise fixedly attached to the proximal end 130 of the coupler 140.

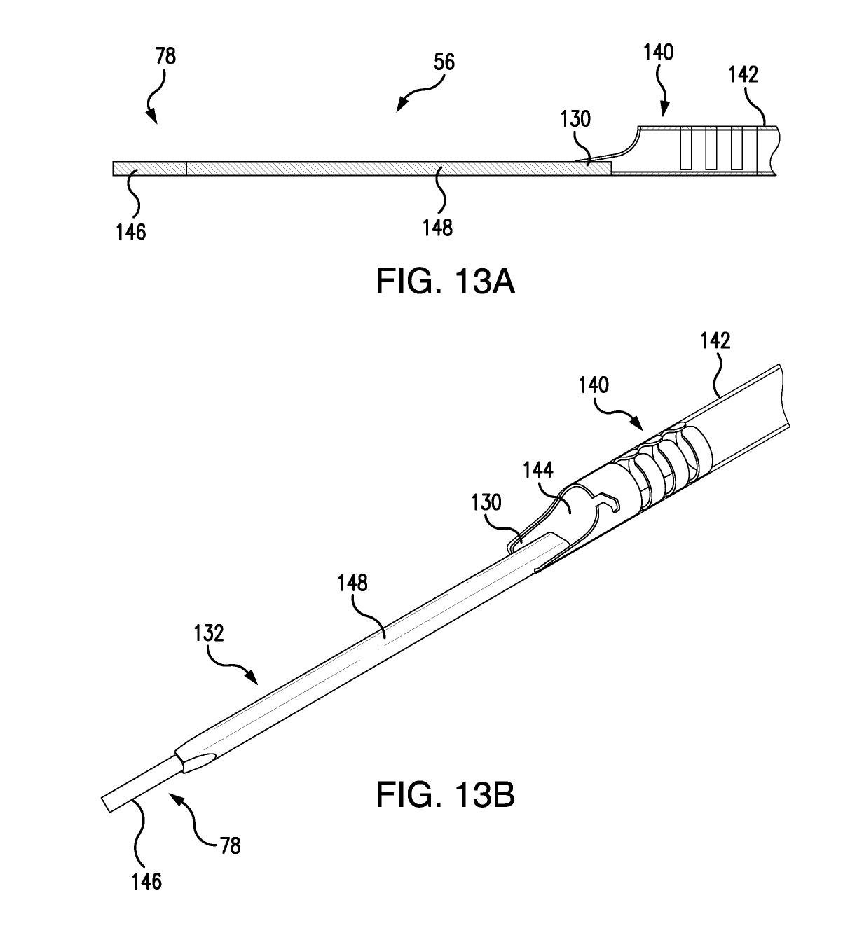

In another implementation of the outer member 56 shown in FIGS. 13A-13B, the round pusher wire 146 can be welded to the flat wire 148 which, in its turn, is welded or otherwise fixedly secured to the proximal end 130 of the sheath 142.

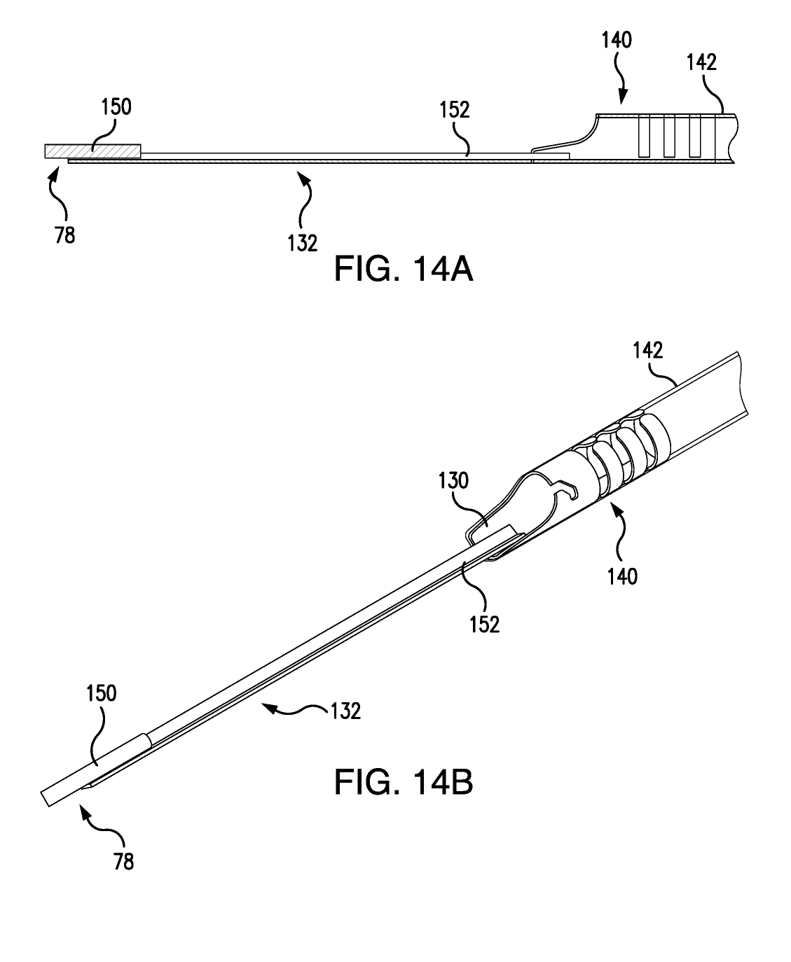

In still another alternative embodiment of the outer member 56, shown in FIGS. 14A-14B, the round wire 150 are welded or otherwise fixedly secured to two flat wires 152, which in their turn, are welded or otherwise fixedly secured to the proximal end 130 of the sheath 142.

The configuration shown in FIGS. 12A-12B, 13A-13B, and 14A-14B provides a flattened profile of the pusher wire portion welded to the coupler 140 of the sheath 142 so that when the member 54 is inserted in the outer member 56, the pusher wire does not create an obstacle for the rotational or longitudinal motion of the inner member 54 inside the coupler 140 and the sheath 142 of the outer member 56, as required by the procedure.

The outer member pusher 132 may be equipped, at the proximal end 154 thereof, with a proximal handle 155 (shown in FIG. 12B) for convenience of a surgeon performing the coronary intervention procedure for manipulation of the outer member 56 to position the outer delivery sheath 142, along with the balloon delivery sub-system 54, at the desired location relative to the lesion 52 in the diseased blood vessel.

In addition, the inner member 54 may be equipped with an inner member pusher 206 (shown in FIGS. 4A and 23A) which may be attached to the inflation hub 76 to facilitate the withdrawal of the inner member 54 from the outer member 56 as required by the coronary intervention procedure, as well as for controlling engagement/disengagement therebetween, for various stages of the cardiac procedure, as will be detailed in further paragraphs. The inner member pusher 206 may be formed with an inner member pusher's handle (not shown in the Drawings) for convenience of a surgeon performing the procedure The handles of the inner and outer members' pushers may be configured with a mechanism (detailed in the U.S. patent application Ser. No. 15/899,603 which is hereby incorporated by reference) which permits an additional releasable locking of the inner and outer members one to another to enhance the integral cooperation thereof in the engaged mode of operation.

The inner member 54 may be either of the over-the-wire configuration or of the RX configuration. In one of the embodiments detailed herein, the guide wire 42 extends through the RX GW port 110 made at the proximal end of the tubular inflation lumen distal shaft 104 into and along the internal channel 120 of the GW lumen 112, as shown in FIGS. 6C-6D, and 7. At the distal section 60 of the subject system 42, the guidewire 50 extends in the GW lumen 112 along the delivery tapered micro-catheter 118, at exits at the distal ends 116 of the GW lumen 112 at the outermost end 72 of the inner member 54, as shown in FIGS. 4A-4B, 8A-8B, 9A-9B, 10A-10B, and 11A-11B.

With respect to FIGS. 4A, 4C and 11A, the outer delivery sheath 142 of the outer member 56 is made with a cylindrically shaped tubular body 166 extending substantially the length of the middle section 62 of the subject system 40. By manipulating the outer member pusher 132, a surgeon actuates the integral advancement of the outer delivery sheath 142 and the inner member 54 along the guide catheter 44. Upon the pre-dilatation procedure has been performed (as will be detailed in further paragraphs), the surgeon controls a required linear backward displacement of the inner member 54 with regard to the sheath 142 of the outer member 56 by manipulating the outer member pusher 132 and/or the inner member pusher 206.

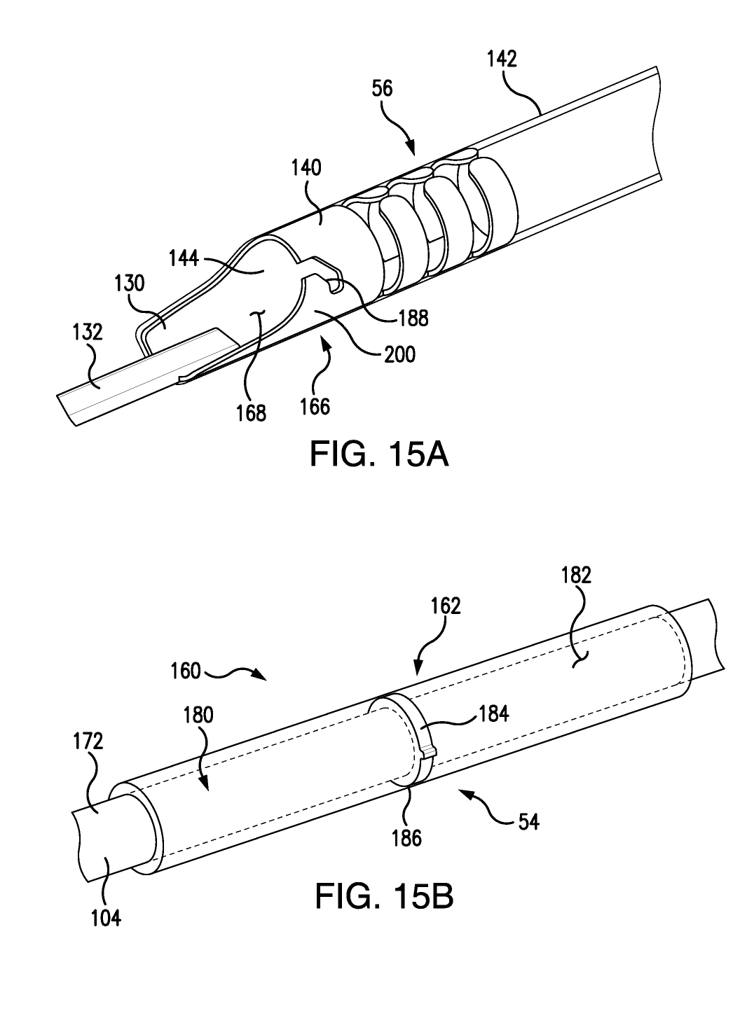

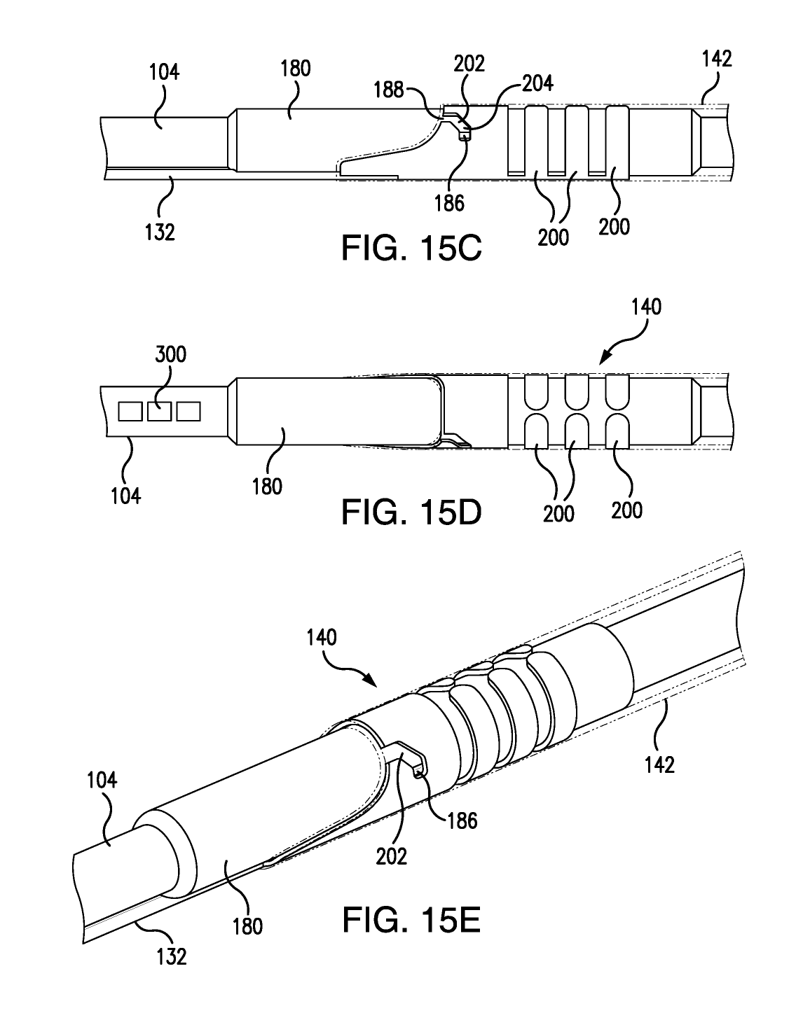

As shown in FIGS. 15A-15E, 16A-16D, 17A-17D, 18A-18D, 19A-19D, 20A-20C, 21A-21B, and 22A-22B, the subject system is built, at the middle section 62, with an interconnection mechanism 160 which includes the coupler 140 formed at the proximal end 130 of the sheath 142 of the outer member 56, and a cooperating mechanism 162 formed at the outer surface of the inner member 54.

The subject guide catheter extension/pre-dilatation system 40 may operate in an inner/outer members engagement mode and in an inner/outer members disengagement mode, which is accomplished by controlling the interconnection mechanism 16. The subject interconnection mechanism is configured to engage/disengage the inner and outer member 54, 56 (as required by the cardiac procedure), as well as to prevent forward displacement of the inner member 54 inside the outer delivery sheath 142. The inner member 54 cannot be advanced forward relative to the outer delivery sheath 42, and can perform exclusively the backward movement for withdrawal from the outer delivery sheath 142.

As shown in FIGS. 4A, 4C and 15A-22B, illustrating the middle section 62 of the subject guide catheter extension/pre-dilatation system 40, the interconnection unit 160 operates with the coupler 140 configured at the proximal end 130 of the sheath 142 and the cooperating mechanism 162 configured at the outer surface 172 of the inner member 54 by interfacing the inner surface 168 of the tubular body 166 of the sheath 142 (at its proximal end 130), with the outer surface 182 of the cooperating mechanism 162 (on the inner member 54) of the interconnection mechanism 160.

There are several interconnection mechanisms envisioned in the subject guide catheter extension/pre-dilatation system 40. The subject engagement mechanism is configured for controllable engagement/disengagement between the inner member 54 and the outer member 56, as well as to prevent a forward motion of the inner member 54 relative the outer delivery sheath 142 beyond a predetermined position.

For example, as shown in FIGS. 4C and 15A-15E, in one of the embodiments, the operation of the interconnection unit 160 is supported by friction-based engagement between the inner surface 168 of the tubular body 166 of the outer delivery sheath 142 and the outer surface 182 of the cooperating mechanism 162 represented by a friction element 180 located at the outer surface 172 of the inner member 54.

As shown in FIGS. 15B-15E, the friction element 180 is a cylindrically shaped member attached (by gluing or welding) to the outer surface 172 of the inner member 54. The friction element 180 may include a lock ring 184 encircling its outer surface at a predetermined location. The lock ring 184 has a locking button 186. In this implementation, as shown in FIGS. 15A-15E, the coupler 140 (located at the proximal end 130 of the cylindrically shaped tubular body 166 of the sheath 142 of the outer member 56) is configured with a coupler lock notch 188 which cooperates with the locking button 186 on the friction element 180 of the inner member 54.

When being disposed in the internal channel 144 of the sheath 142 in frictional cooperation with the ribs 200 of the coupler 140, the locking button 186 on the lock ring 184 is motioned by the surgeon (by manipulating the inner member pusher 206 and/or the outer member pusher 132), first reciprocally along the coupler 140 to enter the coupling lock notch 188, and subsequently rotationally in clockwise direction to move the locking button 186 in the lock notch 188 along the portion 202 to reach the end of the notch 188.

In order to disengage the inner member 54 from the outer member 56, the surgeon removes the locking button 186 from the coupler lock notch 188 by counterclockwise rotation of the lock ring 184 and subsequent removal of the friction element 180 from the coupler 140 of the outer member 56.

In the disengaged configuration, the forward motion of the friction element 180 inside the outer delivery sheath 142 is prevented when the button 186 engages with and is stopped by the proximal edge of the coupler 140.

Another alternative embodiment of the friction-based interconnection mechanism is presented in FIGS. 4A and 4C, where the engagement/disengagement between the inner and outer members 54, 56 is provided by the friction forces between the outer surface 182 of the friction element 180 and the inner surface 168 of the tubular body 166 of the sheath 142. The friction element 180 may have a tapered configuration of its outer surface 182 with the diameter at some portion thereof, for example, its proximal end, exceeding the diameter of the tubular body 166 of the outer delivery sheath 142, to prevent forward motion of the friction element 180 inside the tubular body 166.

The friction mechanism is used in the guide catheter extension/pre-dilatation system 40 to lock the inner member 54 with the outer member 56 (when required by the cardiac procedure) in order to provide the integral displacement of the inner and outer members 54, 56 (by actuating the outer member pusher 132) during the cardiac intervention procedure.

A similar friction-based engagement/disengagement mechanism may be provided at other locations along the length of the inner/outer members interface, for example, at the distal section 60 of the subject system 40.

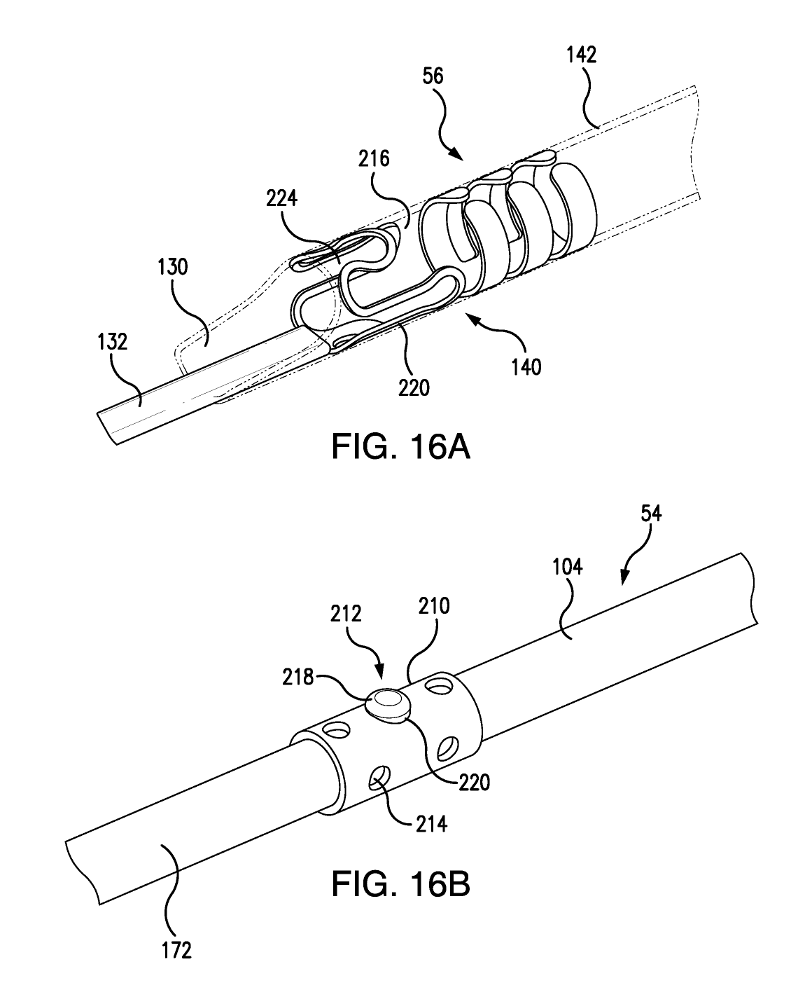

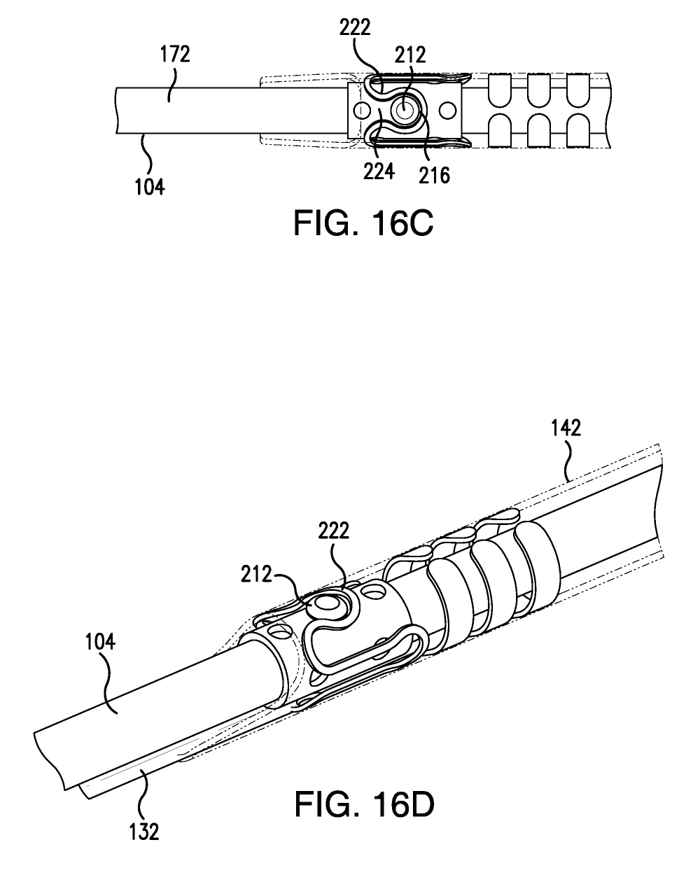

As shown in FIGS. 16A-16D, an alternative embodiment of the subject interconnection mechanism 160 uses a snap-fit “Omega-shape” mechanism, and is configured with the inner member lock band 210 equipped with a snap-fit post 212. The inner member lock band 210 is glued or fused with the outer surface 172 of the inner member 54 and particularly, the inner member's inflation lumen shaft 104. In order to support gluing/fusing of the inner member lock band 210 to the inflation lumen shaft 104, the inner member lock band has glue/fuse ports 214 to introduce adhesive or fusing material thereto.

In order to provide the snap-fit engagement with the snap fit post 212 on the inner member 54, the outer member 56 is configured with an Omega-shaped coupler 216 which includes an Omega-shaped wire (preferably flat wire) at the proximal end 130 of the sheath 142.

In this embodiment, in order to engage the inner and outer members 54, 56, a surgeon linearly displaces the inner member 54 in the internal channel 144 of the cylindrical sheath 142 so that the snap-fit post 212 is entered into the Omega-shaped coupler 216 and is snap-fit therein.

As shown in FIG. 16B, the snap-fit post 212 has an upper circularly portion 218 which is a neck portion 220 which extends between the circularly shaped upper portion 218 and the outer surface of the inner member lock band 210.

When the neck portion 220 of the snap-fit post 212 is engaged with a receptacle formed by the wire 222 of the omega-shaped coupler 216 (as shown in FIGS. 16C-16D), the engagement between the inner and outer members 54, 56 is attained. The height of the neck portion 220 corresponds to the width of the wire 222, while the diameter of the neck portion 220 corresponds to the opening of the entrance channel 224 of the Omega-shaped coupler 216.

The Omega-shaped configuration also prevents the forward displacement of the inner member 54 relative to the outer delivery sheath 142 further than the engagement area of the snap-fit post 212 and the receptacle 224, both in engaged and disengaged modes of operation, since the post 212 (even when outside the receptacle 224) is stopped from forward displacement by the wire 222.

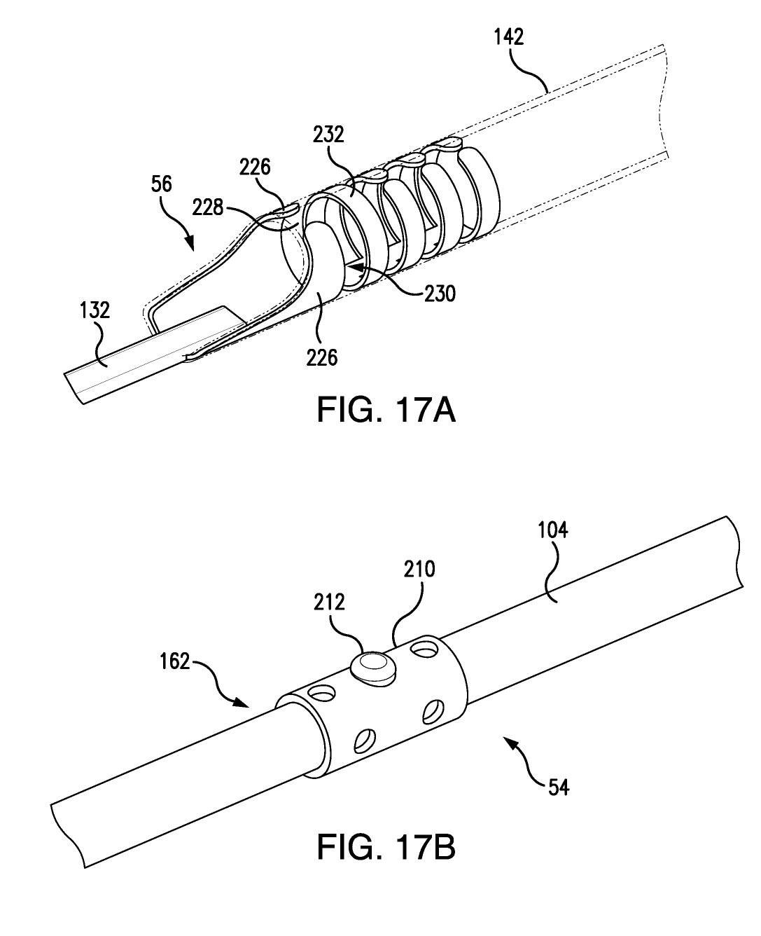

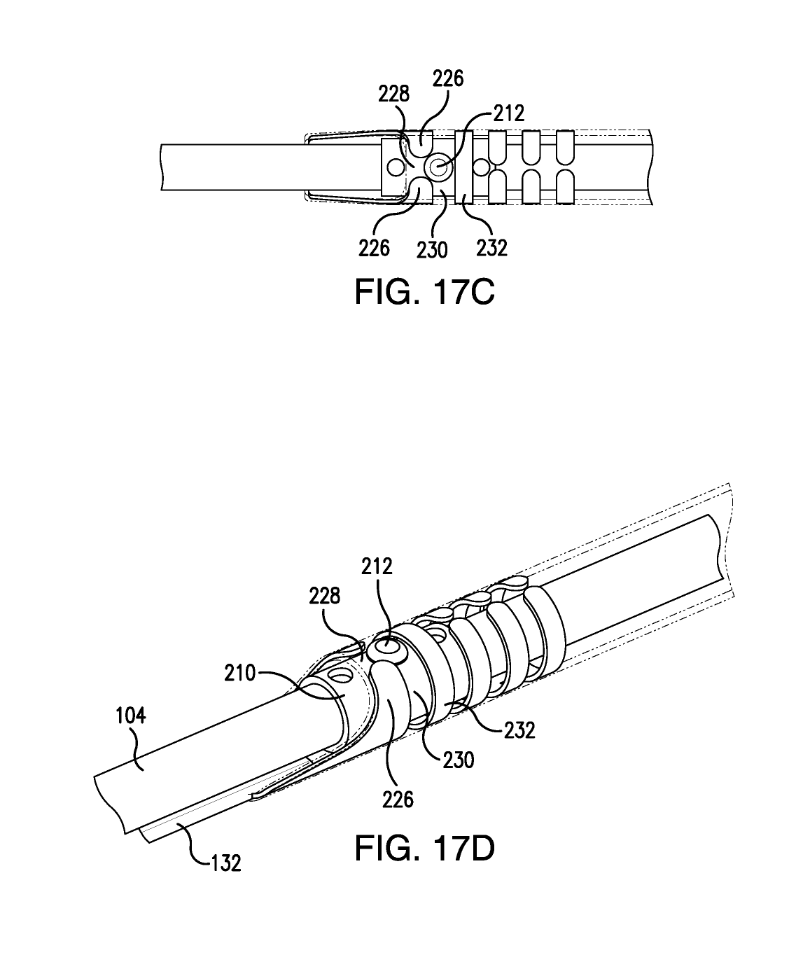

Referring to FIGS. 17A-17D, another alternative snap fit “Simple Rib”-based interconnection mechanism includes the ribs 226 forming at the coupler 140 at the proximal end of the sheath 142. As shown in FIG. 17B, the operating mechanism 162 of the inner member 54 is similar to that one shown in FIG. 16B and will not be further detailed. As shown in FIG. 17C-17D, the snap-fit post 212 on the inner member lock band 210 (at the inner member 54) cooperates with the ribs 226 of the coupler 140 of the outer member 56, thus providing engagement between the inner and outer members 54, 56.

As shown in FIG. 17C, the snap-fit post 212 enters the channel 228 between the ribs 226 and is snap-fit therein. In order to enhance the snap-fit engagement with the ribs 226, the snap-fit post 212 may be displaced either counter-clockwise or clockwise inside the channel 230 between the ribs 226 and the auxiliary rib 232. The auxiliary rib 232 prevents further forward advancement of the post 212, thus preventing the inner member 54 from the forward displacement relative to the sheath 142 in the engaging mode of operation.

For disengagement purposes, the snap-fit post 212 is aligned with the entrance channel 228 and subsequently the inner member 54 is displaced longitudinally with the snap-fit post 212 exiting the entrance channel 228 of the coupler 140 of the outer member 56.

In the disengaging mode of operation, this post 212 is stopped from forward motion by the edges of the ribs 226, thus preventing the forward displacement of the inner member 54 relative the outer delivery sheath 142.

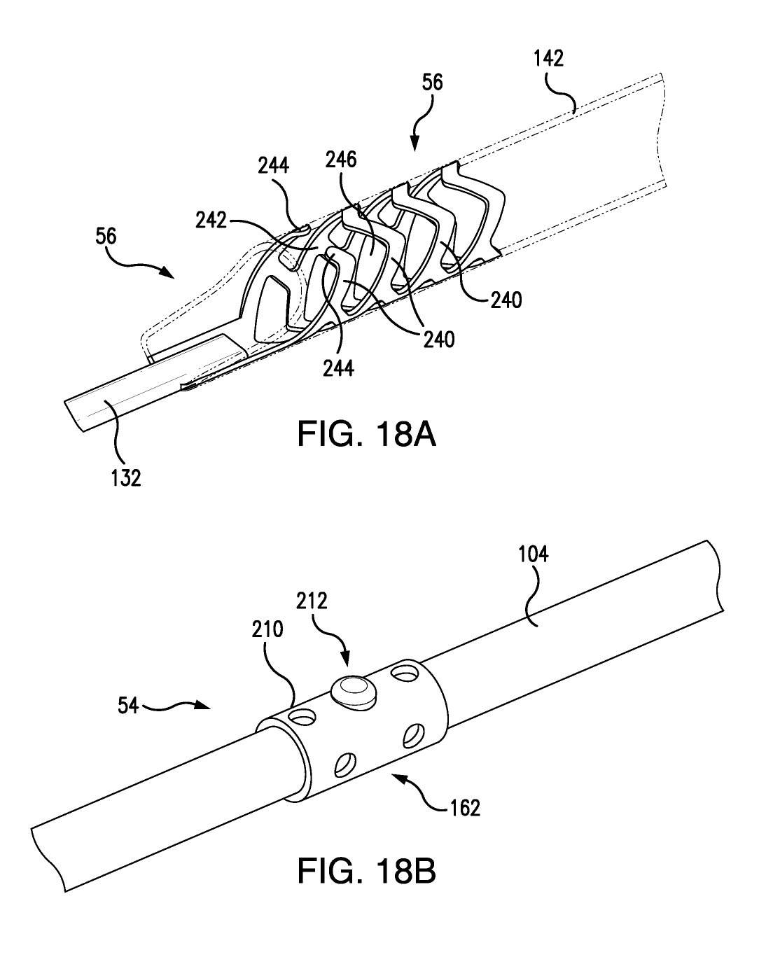

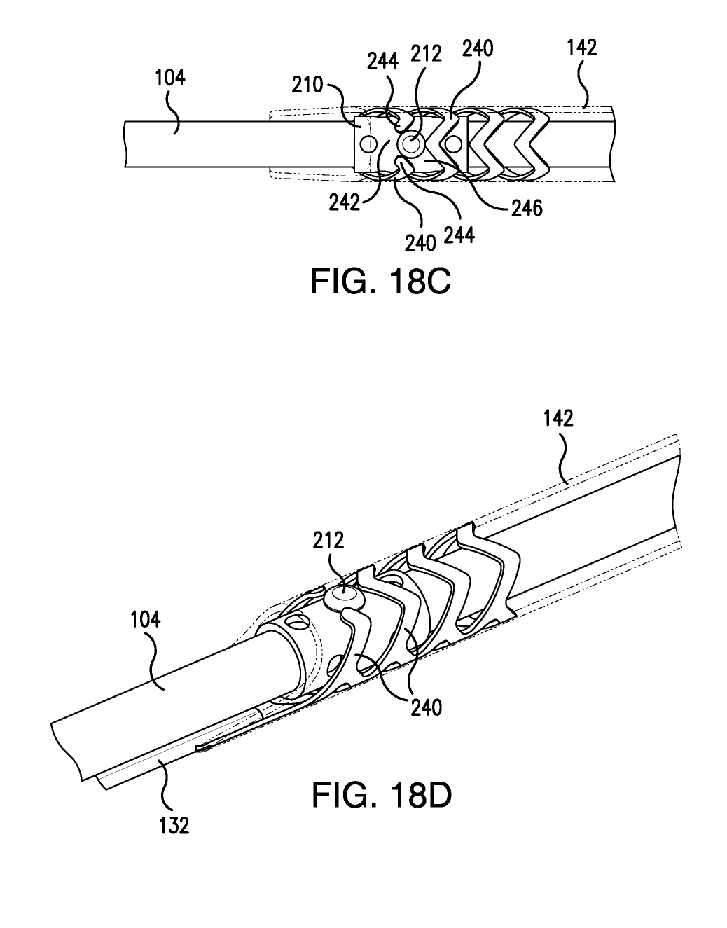

A further embodiment of the interconnection member 160, the snap-fit “W-shape” interconnection mechanism is presented in FIGS. 18A-18D. The inner member's cooperating mechanism 162 of the interconnection mechanism 160 (shown in FIG. 18B) is similar to that described in FIGS. 16B and 17B. However, in the embodiment shown in FIGS. 18A, and 18C-18D, the outer member's coupler 140 is configured with W-shaped ribs 240. In FIGS. 18A, 18C, and 18D, the snap-fit post 212 on the inner member lock-band 210, when displaced by a surgeon, enters an entrance channel 242 configured between the ends 244 of the opposing ribs 240 and is snap-fit there. If the surgeon displaces the snap-fit 212 in clockwise or counter-clockwise direction along the channel 246 formed between the W-shaped ribs 240 and the auxiliary ribs 248, this motion provides enhanced engagement between the inner and outer members 54, 56. Again, similar to the alternative embodiments of the interconnection mechanism 160 presented in previous paragraphs, even in the disengaged mode of operation, the forward motion of the inner member 54 inside the outer delivery sheath 142 is prevented by stopping a forward motion of the post 212 by the edges of the ribs. Only a backward linear motion is permitted for withdrawal of the inner member from the outer delivery sheath 142 in the disengaged mode of operation.

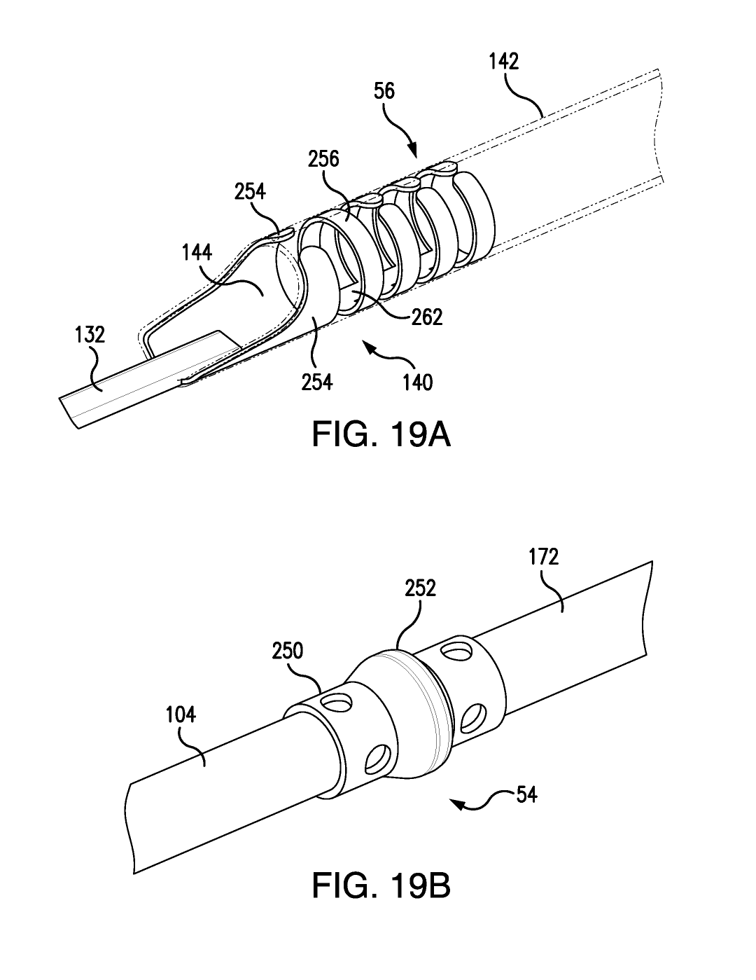

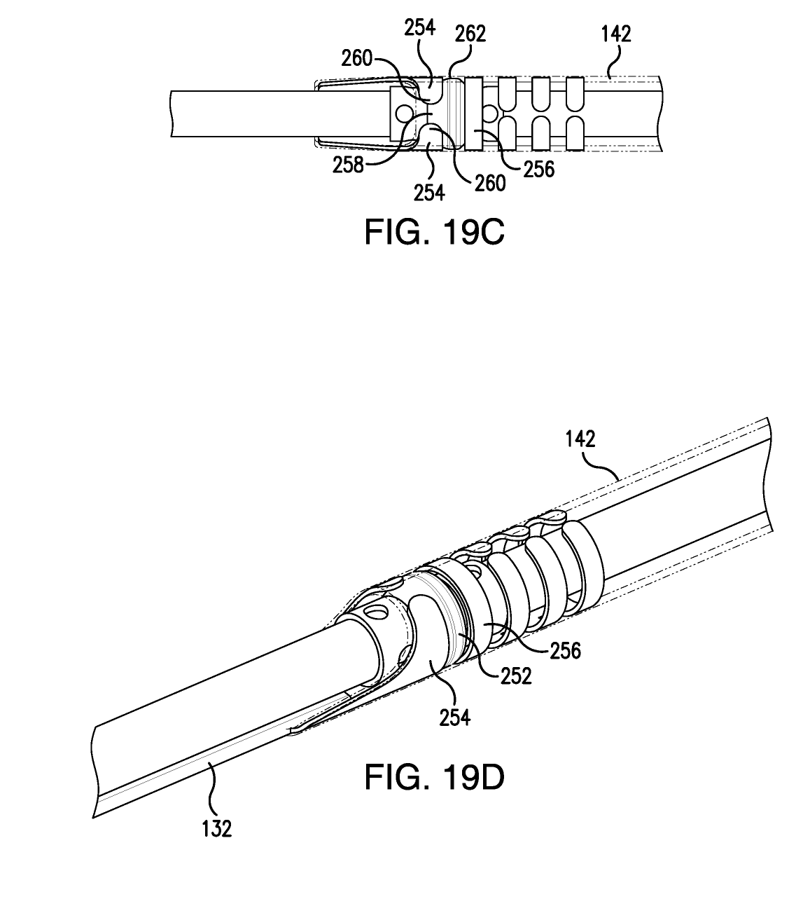

Referring to FIGS. 19A-19D, an alternative circumferential snap-fit interconnection mechanism is shown. As shown in FIG. 19B, the cooperating mechanism 162 of the inner member 54 is represented by the inner member lock band 250 glued/fused or otherwise adhered to the outer surface 172 of the inner member inflation lumen shaft 104. The lock band 250 is configured with a snap-fit annular ring 252. In this embodiment, as shown in FIGS. 19A, 19C, and 19D, the outer member's coupler 140 is configured with the ribs 254 and the secondary rib 256. An entrance channel 258 is formed between the edges 260 of the ribs 254. The secondary rib 256 may be formed as a solid rib to configure a channel 262 between the ribs 254 and the secondary rib 256.

When the surgeon linearly displaces the inner member 54 within the internal channel 144 of the coupler 140, the snap-fit annular ring 252 enters the channel 262 between the ribs 254 and the secondary rib 256. The ribs 254 are flexibly bent outwardly when the snap-fit annular ring passes through the channel 258. When the snap-fit annular ring 252 reaches the channel 262 and aligns therewithin, the ribs 254 return to their original position and snap-fit the snap-fit annular ring 252 within the channel 262.

In order to disengage the inner member 54 from the outer member 56, the surgeon pulls the inner member 54 from the internal channel 144 of the coupler 140. During the removal of the snap-fit annular ring 252 from the channel 262, the pulling action causes the ribs 254 to bend outwardly to permit the passage of the snap-fit annular ring 252 therebetween, thus freeing the inner member lock band 250 from the coupler 140. In the disengaged mode of operation, the snap-fit annular ring 252 stops at the proximal edge of the ribs 260, thus preventing the forward motion of the inner member 54 relative to the outer delivery sheath 142, while in the engaged mode of operation, any linear displacement of the inner member 54 relative to the outer delivery sheath 142 is prevented since the ring 252 is trapped between the ribs 260 and the secondary rib 256.

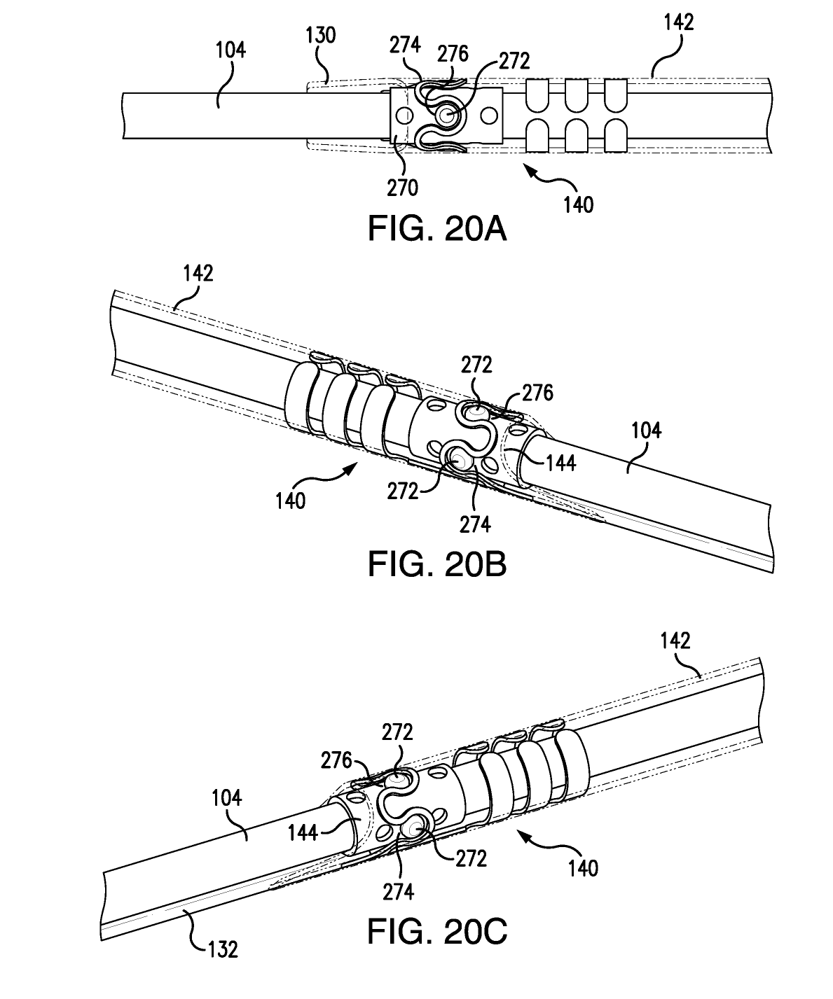

The prevention of the forward displacement of the inner member 54 relative the outer delivery sheath 142 in the disengaged mode of operation or any linear displacement thereof in the engaged mode of operation is also provided by the interconnection mechanism depicted in FIGS. 20A-20C, 21A-21B, and 22A-22B.

In another embodiment, shown in FIGS. 20A-20BC, the snap-fit (three post) 90° orientation interconnection mechanism has the inner member lock band 270 formed with three posts 272 angularly spaced apart substantially 90° around the lock band 270. The Omega-shaped wire 274 is configured with three receptacles 276 to receive the posts 272. Thus, in order to attain the engagement between the inner member 54 and outer member 56, the surgeon longitudinally displaces the inner member 54 within the inner channel 144 of the coupler 140 until the posts 272 are received in respective receptacles 276 and snapped therein. In order to disengage the inner member 54 from the outer member 56, an opposite action is performed by the surgeon.

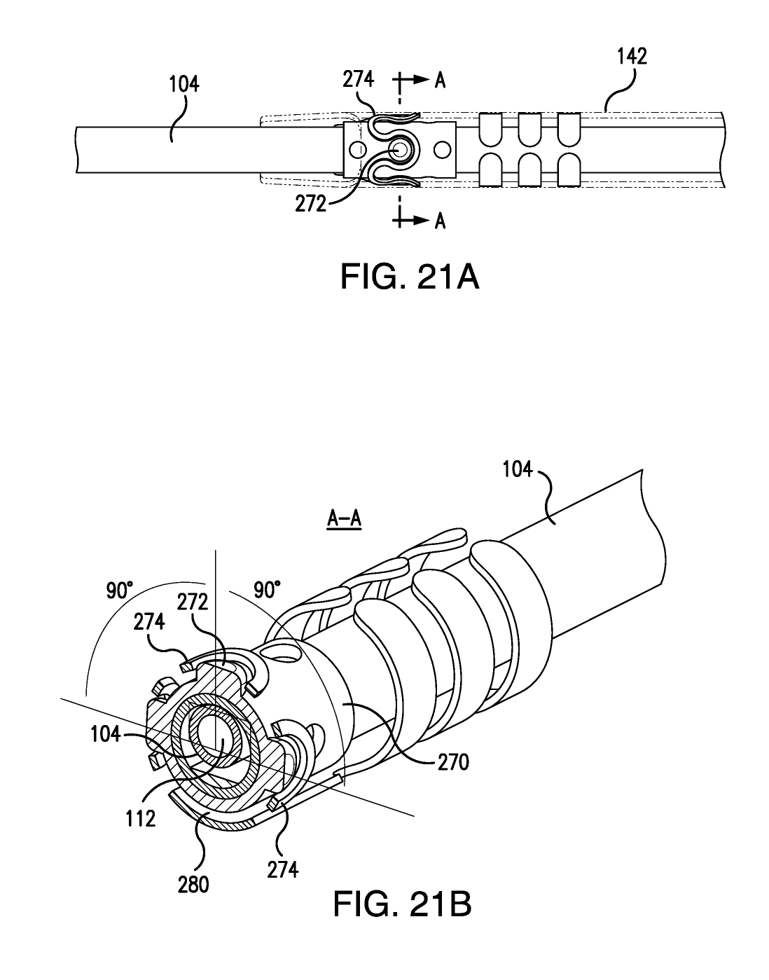

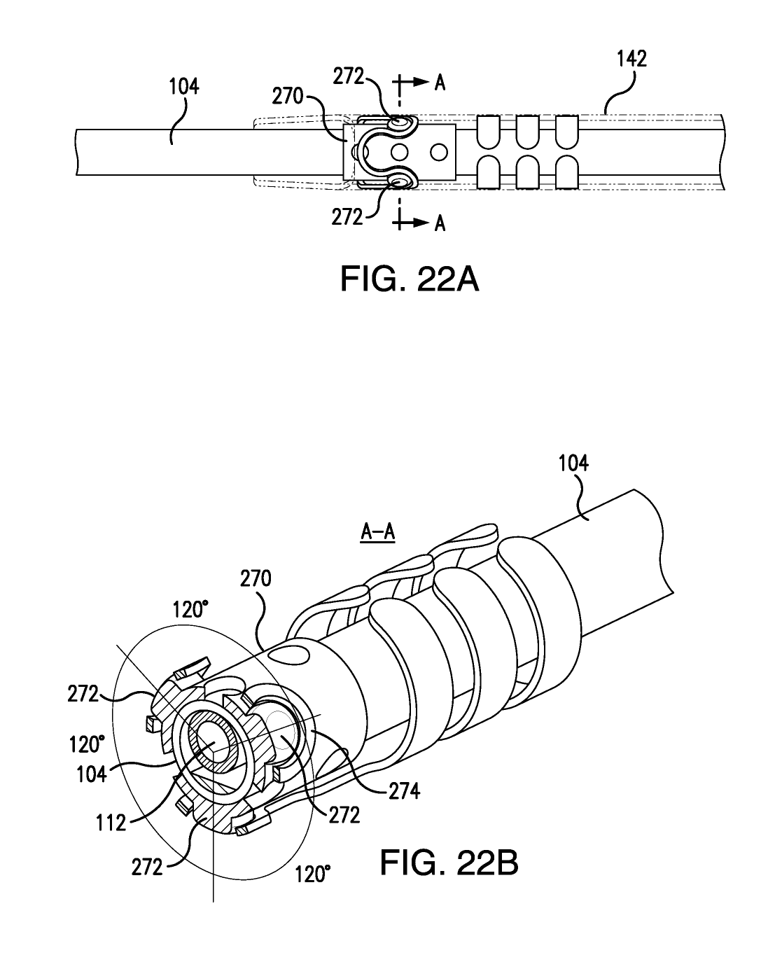

FIGS. 21A-22B detail the arrangement shown in FIGS. 20A-20C. As shown in FIGS. 21A-21B, the GW lumen 112 extends inside the inflation lumen distal shaft 104.

The outer member pusher 132 (as depicted in FIGS. 12A-12B) is tapered (flattened) at its distal end 136, and is welded (glued, adhered, or otherwise fixedly attached) to the proximal end 130 of the tubular body 166 of the sheath 142. The tapered end 136 of the outer member pusher 132 may, alternatively to the flattened configuration, have a somewhat curved low-profile configuration in order to snugly cradle the portion of the outer surface of the interconnection coupling mechanism 162 of the inner member 54 in order to form a smooth surface at their interconnection, as well as to consume as little space within the sheath 142 as possible. The space 280 (shown in FIG. 21B) is provided for the outer pusher wire attachment.

Shown in FIGS. 22A-22B, another alternative embodiment of the subject interconnection mechanism 160 of FIGS. 21A-21B has a snap-fit 3 post arrangement with the 120° angular displacement between the posts 272. The elements of FIGS. 22A-22B are similar to those in FIGS. 21A-21B, and the GW lumen 112 extends inside the inflation lumen distal shaft 104. The difference in the angular spacing between the posts 272 is 120° as compared to 90° angular distance between the posts 272 shown in FIGS. 21A-21B.

The interconnecting mechanism 160 is controlled by a surgeon during the cardiac procedure to disengage the inner member 54 from the outer member 54 when the inner member 54 is to be retracted from the sheath 142 and removed from the guide catheter 44 (as shown in FIG. 23E).

Referring to FIG. 15D, the inflation lumen distal shaft 104 at the middle section 62 of the subject guide catheter/pre-dilatation extension system 40 may be manufactured with a braid reinforcement structure 300. The braid reinforcement member 300 creates a somewhat flexible tubing connected to the cooperating mechanism 162 of the interconnection unit 160 of the inner member 54. The RX (Rapid Exchange) port 110 for passing the guide wire 42 may be formed through the wall of the braid reinforced inflation lumen distal shaft 104, as shown in FIG. 6D.

The braid reinforcement structure 300 may be configured with metallic patterns or wires within the braid reinforced inflation lumen distal shaft 104 to prevent kinking, which would give the shaft 104 a longitudinal stiffness. The metal braid portion 300 may be embedded in the braid reinforced shaft 104 to add increased flexibility thereto required for retraction of the inner member 54 relative to the outer delivery sheath 142 during the procedure.

A flat wire helical coil (made, for example, from a shape memory alloy, such as Nitinol) with a wire thickness of approximately 1 mil to 3 mils may be embedded in the braid portion 300. This coil may be formed with a very thin coating of plastic placed onto its inner and outer surfaces, which facilitates the reduction of the wall thickness of the inflation lumen distal shaft 104 to less than 7 mils and preferably to approximately 5 mils.

The principles of reinforcing the tubular members by a flat wire helical coil 302 or forming the tubular members from the flat wire helical coil may be applied in the subject guide catheter extension/pre-dilatation system 40 to the outer delivery sheath 142, as well as to the micro-catheter 118 (FIG. 8A). In the outer delivery sheath 142 and/or the micro-catheter 118, such flat wire helical coil may be embedded in predetermined positions along the length of the walls thereof, for example, at the proximal and or distal ends.

Alternatively, the entire length of the outer delivery sheath 142 and/or micro-catheter 118 may be formed from the flat wire helical coil. The pitch between the coils may be changed to provide the flexibility gradient along the length of the tubular member (sheath 142 and or micro-catheter 118) increasing towards the distal end thereof to facilitate atraumatic operation. The flat wire helical coil 202 is schematically depicted in FIGS. 4A-4C, 8A, and 11A-11B.

The subject guide catheter extension/pre-dilatation system 40 may be configured with a differential in micro-catheter flexibility with greater flexibility in the distal portion, by either changing the durometer of the plastic components from the outer delivery sheath's proximal portion to its distal portion (i.e., a higher durometer in the proximal portion when taken with respect to the distal portion), and/or changing the winding frequency (pitch) of the helical coil of wire in the micro-catheter 118 in the direction from the proximal portion to distal portion, such that the distal portion of the micro-catheter 106 is more flexible and trackable than the proximal portion of the micro-catheter delivery device, and has a substantially lower profile and is more flexible than the distal portion of the guide catheter extension sub-system (outer delivery sheath).

The system 40 may also include wires that have radio-opacity such that the balloon member 96, micro-catheter 118, and the outer delivery sheath 142 are easily visualized using fluoroscopy. It is envisioned that the distal tip 314 is provided with radio-opaque markers 306, 308 in proximity to the proximal portion 122 and the distal portion 124 of the balloon 96 (as shown in FIGS. 8A, 10A, and 11A-11B). The radio-markers 306, 308 permit the surgeon (operator) to visualize positioning of the balloon member 96 relative to the lesion location 52.

In addition, the outermost distal tip 72 of the micro-catheter delivery portion 118 and the tip 304 of the sheath 142 may have one or more radio-opaque markers 310, 312 (shown in FIGS. 4B-8A) in order to permit the surgeon to distinguish between the radio-markers, which is particularly important as the obstructive lesion is passed by the micro-catheter, and the balloon member carried in proximity to the micro-catheter is held in place.

As shown in FIGS. 4A and 4C, the outer delivery sheath 142 extends between its proximal end 130 at the middle section 62 and its distal end 304 at the distal section 60 of the subject system 40. At the distal section 60 of the subject guide catheter extension/pre-dilatation system 40, the inner member 54 is configured with a tapered distal tip 314 which is formed with the micro-catheter 118. The micro-catheter 118 is an elongated member with the length in a cm range, for example, 1-3 cm. The micro-catheter 118 is a thin member which has a tapered cone-contoured configuration with the diameter not exceeding 1 mm at its distal end 72. The micro-catheter 118 is formed integrally with the tapered distal tip 314 of the inner member 54.

As shown in FIGS. 4A and 8A-8C, at the distal end 304, the outer delivery sheath 142 is formed with an outer tip 316 which has a tapered cone-contoured configuration which may be frictionally (or through an alternative engagement/disengagement mechanism 160 presented in FIGS. 15A-22B) interconnected with the distal tip 314 of the inner member 54. The outer tip 316 of the outer member 56 provides a smooth distal taper transition between the distal end 304 of the sheath 142 and the distal section 60.

In FIGS. 4A, 8, 9A-9B, 10A-10B, 11A-11B, the distal tip 314 is shown to have a tapered configuration which changes gradually from the point of interconnection with the outer tip 316 of the sheath 142 to the distal end 318 of the distal tip 314. The micro-catheter 118 extends from the distal end 318 of the distal tip 314 of the inner member 54 (the length of about 1-3 cm) in an integral connection therewith and terminates in the outermost distal end 72. The diameter of the micro-catheter 118 at the distal end 172 does not exceed 1 mm.

As shown in FIGS. 8A-8C, the pre-dilatation balloon 96 is attached, with its proximal portion 122, to the proximal portion 320 of the distal tip 314 in bordering juxtaposition with the outer tip 316 of the sheath 142, and, with its distal portion 124, to the distal end 318 of the distal tip 314 of the inner member 54.

The distal tip 314 of the inner member 54 at its wider (proximal) diameter has the same dimension as the diameter of the outer tip 316 of the sheath 142 in order to form a substantially smooth outer surface at the distal section 60 of the system 40. An important aspect of the subject system is that for a transition between the outer diameter of the outer tip 316 of the sheath 142 and the outer diameter of the distal tip 314 of the inner member 54 is equal to or less than 0.0004″ to form substantially gradual (smooth) transition therebetween.

The interface between the outer tip 316 of the sheath 142 and the distal tip 314 of the inner member 54 may be chamfered to facilitate displacement of the distal tip 314 of the inner member 54 relative to the outer tip 316 of the sheath 142 and basically to facilitate displacement of the distal tip 314 relative to the outer tip 316 of the sheath 142, as required by the cardiac procedure.

The distal end 304, as well as the outer tip 316 of the sheath 142, are formed of a flexible material which permits an easy retraction of the distal tip 314 of the inner member 54 therethrough. The flat wire helical coil may be used for the distal end 304 and the outer tip 316 of the sheath 142.

The guide wire 42 extends from the proximal section 58 of the subject system 40 through the GW internal lumen 112 formed in the inner member 54, within the sheath 142 and through the distal tip 314 of the inner member 54, and exits at the outermost distal end 72 of the micro-catheter 118 of the inner member 54.

In operation, as shown in FIG. 23A, for performing the cardiac procedure, and specifically the pre-dilatation routine, a proximal end of the coronary guidewire 42 is entered into the RX port 110 formed in the inflation lumen distal shaft 104, and is extended through the inner channel (GW lumen 112) of the inner member 54 towards and beyond the outermost distal end 72 of the micro-catheter 118. Subsequent thereto, the guide catheter 44 is advanced into the blood vessel 45 of interest.

Subsequently, the outer delivery sheath 142 of the outer member 56 locked with the inner member 54 therewithin, are placed first with the micro-catheter 118 in the channel 68 of the guide catheter 44, and both inner and outer members 54, 56 as a single unit, are integrally advanced within the guide catheter 44 towards the treatment site 52. The outer member's sheath 142 and the inner member 54 may be integrally displaced by pushing the outer member pusher 132. This action causes the micro-catheter 118 of the inner member 54 to slide along the GW 42 along with the outer member 56 until they extend beyond the distal end 66 of the guide catheter 44, and reach the lesion site 52, as shown in FIG. 23B. In this step of the procedure, the balloon member 96 is in its deflated configuration.

The guidewire 42 which extends beyond the distal end 66 of the guide catheter 44, serves as a guide along which the micro-catheter 118 (with the deflated balloon 96 attached to the distal tip 314) slides towards the treatment site 52.

Subsequently, as shown in FIG. 23C, the balloon member 96 (which is positioned at the treatment site 52) is inflated by the balloon inflation system 95 connected to the inflation hub 76 through the inflation lumen formed by the inflation lumen distal shaft 104 and the inflation lumen hypotube 88 in order to compress the plaque and to widen the blood passage inside the blood vessel 45.

Subsequently, once the lesion has been dilated, as shown in FIG. 23D, the balloon 96 is deflated, and the outer delivery sheath 142 may be advanced across the lesion 52 either as an integral unit with the inner member 54 (in the engaged mode of operation), and the inner member may be subsequently disengaged (unlocked) from the outer delivery sheath 142 and removed from the sheath 142 (as shown in FIG. 23E).

Alternatively, the inner member 54 may be disengaged and withdrawn from the sheath 142 directly after the lesion dilatation, while the outer member 56 is advanced across the lesion 52 (as shown in FIG. 23E).

The sheath 42 may be left in place (directly after the dilatation of the lesion) proximal to the treatment site, as shown in FIG. 23E.

Subsequent to pulling the inner member 54, the stent 200 can be delivered to the site 52. As shown in FIG. 23F, the stent 200, in its closed configuration, may be introduced into the blood vessel 45 inside the sheath 142. When in place, the stent supporting balloon (not shown) may be expanded, thus opening the stent. Subsequently, the outer delivery sheath 142 is removed, leaving the opened stent in the blood vessel 45.

Although this invention has been described in connection with specific forms and embodiments thereof, it will be appreciated that various modifications other than those discussed above may be resorted to without departing from the spirit or scope of the invention as defined in the appended claims. For example, functionally equivalent elements may be substituted for those specifically shown and described, certain features may be used independently of other features, and in certain cases, particular locations of elements, steps, or processes may be reversed or interposed, all without departing from the spirit or scope of the invention as defined in the appended claims.

What is claimed is:

1. An intravascular delivery system configured for controllable displacement along a guide wire in a blood vessel of interest, comprising:
a proximal section, a distal section, and a middle section portion positioned between said proximal and middle sections,
an outer member formed by a flexible substantially cylindrically contoured elongated outer delivery sheath defining a sheath lumen having a proximal end and a distal end, said outer delivery sheath extending between said middle section and distal section and is configured with a tapered outer tip at said distal end of said sheath lumen;
an inner member having an elongated body defining an internal channel extending along the longitudinal axis thereof, said inner member extending internally along said sheath lumen of said outer member in a controllable relationship with said outer delivery sheath, wherein said inner member has a tapered distal tip configured with a tapered delivery micro-catheter having an elongated body of a predetermined length, said tapered delivery micro-catheter being displaceable along said guide wire beyond said distal end of said sheath;
a balloon member attached to said tapered distal tip of said inner member in proximity to said tapered delivery micro-catheter;
an inflation lumen extending inside said inner member between said proximal section and said balloon member at said distal section to provide a fluid passage between a balloon inflation system and said balloon member; and
an interconnection mechanism disposed in an operative coupling with said inner and outer members and controllably actuated to operate said guide catheter extension/pre-dilatation sub-system in an engaged or disengaged modes of operation, wherein said interconnection mechanism is configured to prevent a forward displacement of said inner member relative to said outer member;
wherein, in said engaged mode of operation, said inner and outer members of said guide catheter extension sub-system are engaged for a controllable common displacement along the guide wire, and
wherein, in said disengaged mode of operation, said inner and outer members are disengaged for retraction of said inner member from said outer member subsequent to the pre-dilatation treatment.

2. The intravascular system of claim 1, wherein said balloon member has a proximal portion having a diameter exceeding a diameter at a distal portion thereof.

3. The intravascular system of claim 1, wherein said balloon member assumes an inflated configuration and a deflated configuration, wherein in said deflated configuration, said balloon member is displaced in the blood vessel, and wherein when said balloon member is controllably transformed into said inflated configuration subsequently to being positioned at least in alignment with the treatment site for the pre-dilatation procedure.

4. The intravascular system of claim 1, wherein said predetermined length of said micro-catheter is in a cm range.

5. The intravascular system of claim 1, wherein a diameter of said micro-catheter at a distal end thereof does not exceed 1 mm.

6. The intravascular system of claim 1, wherein said outer delivery sheath, at the distal end thereof, is configured with a tapered outer tip,
wherein said tapered distal tip of said inner member interfaces, at the outer surface thereof, with an inner surface of said tapered outer tip of said sheath lumen, and
wherein a dimensional transition between the outer diameter of said outer tip of said sheath lumen and the outer diameter of said distal tip of said inner member is below 0.004″, thus forming a substantially flush interface transition therebetween.

7. The intravascular system of claim 1, further comprising:
an outer member pusher configured with a flattened portion at a distal end thereof and secured to said proximal end of said outer delivery sheath of said outer member.

8. The intravascular system of claim 7, wherein said inflation tube includes:
an inflation lumen hypotube coupled, by a proximal end thereof, to the balloon inflation system and configured with a skived portion at a distal end thereof, and
an inflation lumen distal shaft having a proximal end overlapping with said skived portion at the distal end of said inflation lumen hypotube, and a distal end extending towards said balloon member and coupled thereto in fluidly sealed communication therewith.

9. The intravascular system of claim 1, wherein said interconnection mechanism is a friction-based unit interfacing an outer surface of said inner member and an inner surface of said outer delivery sheath of said outer member.

10. The intravascular system of claim 9, wherein said friction-based interconnection unit includes at least one engagement button extending above an external surface of said inner member, and at least one engagement slot configured at least at said proximal end of said outer delivery sheath of said outer member, wherein in said engaged mode of operation, said at least one engagement button is removably engaged in said at least one engagement slot for locking said inner and outer members one to another.

11. The intravascular system of claim 9, wherein said friction-based unit includes a cylindrically shaped outer surface having a longitudinally tapered configuration having at least one portion with a diameter exceeding a diameter of said outer delivery sheath.

12. The intravascular system of claim 1, wherein said interconnection mechanism includes a snap-fit mechanism, said snap-fit mechanism being configured with at least one snap-fit post formed at said inner member and extending above an external surface thereof.

13. The intravascular system of claim 12, wherein said snap-fit mechanism further includes an outer member coupler configured at said proximal end of said outer delivery sheath of said outer member, and cooperating with said at least one snap-fit post configured on said inner member, said outer member coupler including at least one coupling unit configured to releasably engage said at least one snap-fit post therein, and selected from a group comprising at least two arcuatedly configured ribs, at least a pair of W-shaped ribs, and at least one Omega-shaped element.

14. The intravascular system of claim 13, further including a snap-fit annular ring secured to said external surface of said inner member in an encircling relationship therewith, said snap-fit annular ring cooperating with said arcuatedly configured ribs of said outer member.

15. The intravascular system of claim 12, wherein said snap-fit mechanism includes a plurality of said snap-fit posts angularly spaced apart around said inner member.

16. The intravascular system of claim 11, wherein said micro-catheter is formed of a flexible material having differential flexibility along the length thereof, wherein the flexibility of said micro-catheter increases towards the distal end thereof.

17. The intravascular system of claim 16, wherein said micro-catheter includes a flat wire helical coil extending along said predetermined length of said micro-catheter, and wherein the pitch of said flat wire helical coil changes along the length of said micro-catheter to increase the flexibility of the micro-catheter towards the distal end thereof.

18. The intravascular system of claim 1, further including a flat wire helical coil member forming at least a portion of respective walls of a member selected from a group including said outer delivery sheath of said outer member and said micro-catheter.

19. The intravascular system of claim 7, wherein said outer member pusher is selected from a group comprising: a round solid wire flattened at the distal end thereof, a round wire welded to a flat wire, and a round wire welded to a pair of flat wires.

20. The intravascular system of claim 18, wherein said flat wire helical coil is formed with a shape memory alloy including Nitinol.

21. The intravascular system of claim 18, wherein said flat wire helical coil is formed of a radio-opaque material.

22. The intravascular system of claim 1, further including radio-opaque markers attached to at least said distal end of said outer delivery sheath and a distal end of said micro-catheter.

23. The intravascular system of claim 1, further including radio-opaque markers attached to said tapered distal tip of said inner member in proximity to said proximal and distal portions of said balloon member.

24. A method for intravascular treatment using a guide catheter extension/pre-dilatation system, comprising the steps of:
(a) assembling a guide catheter extension system having:
an outer member formed by a flexible substantially cylindrically contoured elongated outer delivery sheath defining a sheath lumen having a proximal end and a distal end,
an inner member having an elongated body defining an internal channel extending along the longitudinal axis thereof, wherein said inner member extends inside said sheath lumen of said outer member and has a tapered distal tip configured with a tapered delivery micro-catheter having an elongated body of a predetermined length and a balloon member attached to said tapered distal tip in proximity to said micro-catheter, wherein said internal channel supports a fluid communication between said balloon member and a balloon inflation system for controllable inflation/deflation of said balloon member between an inflated and deflated configurations thereof; and
an interconnection mechanism disposed in an operative coupling with said inner and outer members, and controllably actuated to operate said guide catheter extension/pre-dilatation system in an engaged or disengaged modes of operation, said interconnection mechanism being configured to prevent a forward linear displacement of said inner member relative to said outer member;
wherein, in said engaged mode of operation, said inner and outer members of said guide catheter extension system are engaged for a controllable integral displacement in a blood vessel, and
wherein, in said disengaged mode of operation, said inner and outer members are disengaged for a controllable retraction of said inner member from said outer member;
(b) extending a guide wire along said internal channel of said inner member with a proximal end of the guide wire extending outside of said inner member at a proximal end thereof, and a distal end of the guide wire extending beyond a distal end of said delivery micro-catheter;
(c) advancing the distal end of the guide wire into a blood vessel of interest towards a treatment site, and sliding a guide catheter in the blood vessel along the guide wire;
(d) controlling said interconnection mechanism to establish said engaged mode of operation;
(e) advancing said inner and outer members engaged together along the blood vessel of interest, with said balloon member in the deflated configuration thereof, by pushing said outer member, thus causing said micro-catheter to slide along the guide wire towards the treatment site until said balloon member attached to said tapered distal tip of said inner member is being brought in alignment with the treatment site;
(f) inflating said balloon member for the pre-dilatation;
(g) deflating said balloon member subsequent to the pre-dilatation;
(h) advancing said outer member across the pre-dilatated lesion;
(i) controlling said interconnection mechanism to switch to said disengaged mode of operation; and
(j) withdrawing said inner member from said outer member.

25. The method of claim 24, further comprising the steps of:
subsequent to said step (j),
advancing a stent system to the treatment site inside said outer delivery sheath of said outer member remaining inside the guide catheter, and
removing said outer delivery sheath from the guide catheter upon deployment of the stent has been attained.

26. The method of claim 24, further comprising:
coupling an outer member pusher, at a distal end thereof, to said proximal end of said outer member, and
controlling displacement of said outer member by actuating said outer member pusher.

27. The method of claim 22, further comprising:
in step (a), configuring said interconnection mechanism as a friction-based interface between an outer surface of said inner member and an inner surface of said sheath of said outer member.

28. The method of claim 24, further comprising:
in said step (a), configuring said interconnection mechanism with a snap-fit engagement/disengagement mechanism including at least one snap post extending above an external surface of said inner member, and at least one engagement channel formed at the proximal end of said sheath of said outer member, and
in said steps (d) and (i), actuating said outer member pusher to result in engaging/disengaging of said at least one snap post with said at least one engagement channel.

29. The method of claim 24, further comprising:
reconfiguring said elongated body of said inner member at a reinforced portion thereof with a flat wire helical coil embedded in a wall of said elongated body and extended circumferentially around said internal channel of said elongated body.

30. The method of claim 29, further comprising:
forming said flat wire helical coil with a shape memory alloy including Nitinol.

31. The method of claim 29, further comprising:
forming said flat wire helical coil of a radio-opaque material.

32. The method of claim 24, further comprising:
marking said distal end of said sheath and a distal end of said micro-catheter with radio-opaque markers.

33. The method of claim 24, further comprising:
marking a proximal portion and a distal portion of said balloon member with radio-opaque markers.

34. The method of claim 29, further comprising:
forming said micro-catheter with a differential flexibility along the length thereof, wherein the flexibility increases towards said distal end of said micro-catheter.

35. The method of claim 34, further comprising:
extending said flat wire helical coil along the length of said micro-catheter, and
changing the pitch of said flat wire helical coil along the length of said micro-catheter to increase flexibility of the micro-catheter towards said distal tip thereof.

36. The method of claim 24, further comprising:
forming said micro-catheter with a length in a cm range and a diameter at said distal end thereof below 1 mm.

37. The method of claim 24, further comprising:
configuring said outer delivery sheath, at the distal end thereof, with a tapered outer tip,
interfacing said tapered distal tip of said inner member, at the outer surface thereof, with an inner surface of said tapered outer tip of said outer delivery sheath, and
forming a dimensional transition between the outer diameter of said outer tip of said outer delivery sheath and the outer diameter of said distal tip of said inner member below 0.004″, thus forming a substantially flush transition therebetween.
